(19) United States
(12) Patent Application Publication
(10) Pub. No.: US 2026/0327598 A1
(43) Pub. Date: Oct. 22, 2026

(54) METHODS, DEVICES, AND SYSTEMS FOR INTRACELLULAR UPTAKE OF MOLECULES VIA MODULATION OF WAVEFORM CHARGE BALANCE

(71) Applicant: North Carolina State University, Raleigh, NC (US)

(72) Inventors: Michael Benjamin Sano, Chapel Hill, NC (US); Robert H. Williamson, Holly Springs, NC (US); Jordan Fong, Raleigh, NC (US); Alexia Cash, Mooresville, NC (US)

(21) Appl. No.: 19/326,869

(22) Filed: Sep. 12, 2025

Related U.S. Application Data
(60) Provisional application No. 63/695,059, filed on Sep. 16, 2024.

(51) Int. Cl. C12N 13/00

(52) U.S. Cl. CPC C12N 13/00

(57) ABSTRACT

Example embodiments of the present disclosure provide methods, devices, and systems involving the use of electrical pulses to permeabilize cells and tissues to increase uptake of molecular components to elicit cellular responses.

### RELATED APPLICATIONS

The present application claims the benefit of U.S. Provisional Patent Application Ser. No. 63/695,059, entitled “METHODS, DEVICES, AND SYSTEMS FOR INTRACELLULAR UPTAKE OF MOLECULES VIA MODULATION OF WAVEFORM CHARGE BALANCE,” filed on Sep. 16, 2024, with the United States Patent and Trademark Office, the disclosure of which is hereby incorporated by reference herein in its entirety.

### STATEMENT OF GOVERNMENT INTEREST

This invention was made with government support under grant numbers CA272550, CA275587, and CA276232 awarded by the National Institutes of Health. The government has certain rights in the invention.

### BACKGROUND

The present disclosure generally relates to methods, devices, and systems involving the use of electrical pulses to permeabilize cells and tissues to increase uptake of molecular components.

Electroporation may be a physical method of facilitating intracellular delivery of impermeable molecular components. By applying an external electric field via electrical pulses, the cell membrane may be charged to a point where nanoscale pores develop in the membrane, thereby increasing cell permeability. This may enable transmembrane transport of a variety of molecules, such as, for example, large membrane-impermeable molecules (e.g., DNA). Electroporation may be utilized in numerous areas, such as, for example, in vitro and in vivo non-viral nucleic acid delivery (transfection), as well as drug delivery (e.g., electrochemotherapy), biomanufacturing, and gene therapy.

### SUMMARY

According to some aspects of the present disclosure, a method for transiently enhancing transport of molecules across a cell membrane comprises coupling one or more electrodes to target cells or tissue, administering one or more target molecules to an environment surrounding the target cells or tissue, and delivering an asymmetric charge-balanced electrical waveform to the target cells or tissue via the one or more electrodes, the asymmetric charge-balanced electrical waveform comprising a plurality of electrical pulses, wherein respective ones of the electrical pulses comprise first and second phases separated by an interphase delay, the first and second phases having opposite polarities, wherein successive ones of the electrical pulses are separated by an interpulse delay, and wherein at least some of the first and second phases are transiently asymmetric but result in a net charge balance for the asymmetric charge-balanced electrical waveform by an end of delivery.

In some embodiments, the asymmetric charge-balanced electrical waveform comprises at least one of a rectangular waveform including rectangular electrical pulses or a sinusoidal waveform including sinusoidal electrical pulses.

In some embodiments, the respective ones of the electrical pulses comprise bipolar square waves, a respective duration of the first phase is between 10 nanoseconds and 10 microseconds, and a respective duration of the second phase is between 10 nanoseconds and 10 microseconds.

In some embodiments, the respective duration of the first phase and the respective duration of the second phase are each between 0.5 and 2 microseconds, and the interphase delay is between 0.5 and 2 microseconds.

In some embodiments, delivery of the asymmetric charge-balanced electrical waveform induces reversible electroporation in the target cells or tissue, and at least some of the first and second phases are different in duration and/or magnitude.

In some embodiments, the method further comprises placing one or more sensing probes in proximity to the one or more electrodes, and adjusting the interpulse delay between successive ones of the electrical pulses while delivering the asymmetric charge-balanced electrical waveform to the target cells or tissue, based on data from the one or more sensing probes.

In some embodiments, the one or more sensing probes measure a temperature of the target cells or tissue, and the interpulse delay is adjusted based on the data from the one or more sensing probes to elevate the temperature of the target cells or tissue above 37° C. but below 45° C.

In some embodiments, the one or more target molecules comprise at least one of a recombinant nucleic acid, a nucleic acid intended for the target cells or tissue to manufacture an antigen which acts as a vaccine, a nanoparticle, a therapeutic drug substance, an immunotherapy drug substance, or a gene therapy drug substance.

In some embodiments, the one or more target molecules comprise a nucleic acid that results in manufacture of a therapeutic drug substance responsive to transport across a cell membrane of the target cells or tissue.

In some embodiments, the one or more target molecules comprise multiple different nucleic acids which result in production of molecules that cannot be created from a single nucleic acid responsive to transport across a cell membrane of the target cells or tissue.

In some embodiments, transport of the one or more target molecules across a cell membrane of the target cells or tissue results in a biological manufacturing of at least one of: a virus-like particle (VLP), a protein-based therapeutic substance comprising at least one of a protein, a peptide, an antibody, or an enzyme, a nucleic acid-based drug substance comprising at least one of DNA, a plasmid, an oligonucleotide, an aptamer, a DNAzyme, an RNA aptamer, an RNA decoy, a microRNA fragment, or a small interfering RNA fragment, or a gene therapy drug substance.

In some embodiments, a respective duration of the first phase is at least twice as long as a respective duration of the second phase, and the respective duration of the first phase is at least twice as long as a respective duration of the interphase delay.

In some embodiments, a respective duration of the first phase is longer than a respective duration of the second phase.

In some embodiments, the respective duration of the first phase is 2 microseconds or less, and the respective duration of the second phase is 1 microsecond or less.

In some embodiments, a respective duration of the interphase delay is 1 microsecond or less.

In some embodiments, a respective duration of the interphase delay is substantially equal to the respective duration of the second phase.

In some embodiments, the asymmetric charge-balanced electrical waveform comprises a sinusoidal waveform including sinusoidal electrical pulses, and the first and second phases of a first one of the electrical pulses are sinusoidal phases that have different durations and a same magnitude.

In some embodiments, the asymmetric charge-balanced electrical waveform comprises a sinusoidal waveform including sinusoidal electrical pulses, the first phase of a first one of the electrical pulses is followed by the second phase of the first one of the electrical pulses, the first phase of a second one of the electrical pulses is followed by the second phase of the second one of the electrical pulses, the second phase of the first one of the electrical pulses is followed by the first phase of the second one of the electrical pulses, without any remaining ones of the electrical pulses therebetween, and the second phase of the first one of the electrical pulses has a same magnitude as the first phase of the second one of the electrical pulses.

In some embodiments, the second phase of the first one of the electrical pulses has a same duration as the first phase of the second one of the electrical pulses.

In some embodiments, the second phase of the first one of the electrical pulses has a different duration from the first phase of the second one of the electrical pulses.

In some embodiments, the asymmetric charge-balanced electrical waveform comprises at least one of a ramp waveform including ramp electrical pulses, an exponential decay waveform including exponential decay electrical pulses, or a sinusoidal waveform including both a sinusoidal electrical pulse and a trapezoidal electrical pulse.

In some embodiments, at least some of the some of the first and second phases are different in magnitude, and a magnitude of at least one of the electrical pulses is in a range of 500 V/cm to 1500 V/cm.

According to some aspects of the present disclosure, an apparatus configured to transiently enhance transport of molecules across a cell membrane comprises one or more electrodes configured to deliver an asymmetric charge-balanced electrical waveform to target cells or tissue, the asymmetric charge-balanced electrical waveform comprising a plurality of electrical pulses, a power supply configured to output the electrical pulses to the one or more electrodes, and a controller communicatively coupled with the power supply and configured to control the power supply to output the electrical pulses, wherein respective ones of the electrical pulses comprise first and second phases separated by an interphase delay, the first and second phases having opposite polarities, wherein successive ones of the electrical pulses are separated by an interpulse delay, and wherein at least some of the first and second phases are transiently asymmetric but result in a net charge balance for the asymmetric charge-balanced electrical waveform by an end of delivery.

In some embodiments, the controller comprises programming for independent selection of respective durations and magnitudes of the first and second phases.

In some embodiments, the one or more electrodes are further configured to administer one or more target molecules to an environment surrounding the target cells or tissue.

According to some aspects of the present disclosure, a method for transiently enhancing transport of molecules across a cell membrane using electrical pulses comprises coupling one or more electrodes to target cells, administering one or more target molecules to an environment surrounding the target cells, and delivering a waveform comprising a plurality of electrical pulses to the target cells via the one or more electrodes, wherein the electrical pulses comprise cycles of positive and negative phases separated by an interphase delay, wherein ones of the electrical pulses are separated by an interpulse delay, and wherein delivering the waveform is performed in vitro, in vivo, or ex vivo.

In some embodiments, the electrical pulses comprise at least one of a rectangular pulse, a ramp pulse, a decaying exponential pulse, a sinusoidal pulse, a triangular pulse, or a step pulse, and the electrical pulses are unipolar or bipolar.

In some embodiments, the electrical pulses comprise bipolar square waves and a length of each of the electrical pulses is 10 microseconds or less, and an amplitude and a duration of a single phase of the electrical pulses are selected so as to administer electroporation to electrically excitable tissue with reduced stimulation of the tissue as compared to higher amplitude and longer duration pulse protocols.

In some embodiments, the waveform comprises individual cycles including a positive phase and a negative phase separated by the interphase delay, and each of the cycles is separated by the interpulse delay.

In some embodiments, the positive phase and the negative phase are equivalent in duration and magnitude to achieve an overall neutral charge.

In some embodiments, the positive phase and the negative phase are different in duration and/or magnitude to achieve an overall non-neutral charge.

In some embodiments, the waveform comprises a plurality of cycles separated by an intercycle delay creating one or more bursts of pulses, and each burst is separated by the interpulse delay.

In some embodiments, the plurality of cycles have phases of equivalent duration and magnitude to achieve a neutrally charged burst.

In some embodiments, the plurality of cycles have phases of different durations and/or magnitudes to achieve a non-neutrally charged burst.

In some embodiments, the plurality of cycles have phases of different durations and/or magnitudes which are flipped with each successive cycle to achieve an overall neutrally charged burst.

In some embodiments, the plurality of cycles have phases of different durations and/or magnitudes such that the cycles in a first half of the burst have a corresponding cycle in a second half of the burst where the phase durations and/or magnitudes are flipped to achieve an overall neutrally charged burst.

In some embodiments, one or more probes are inserted in the proximity of the one or more electrodes before the electrical pulses are delivered, and the one or more probes are used to adjust the interpulse delay of the electrical pulses while delivering the electrical pulses to the target cells.

In some embodiments, the mechanism of transport enhancement is temperature regulated electroporation, the one or more probes measure temperature, and the one or more probes are used to adjust the interpulse delay to ensure the target cells are not heated beyond a predetermined set point.

In some embodiments, the one or more transported molecules include at least one of a nucleic acid intended for cells to manufacture an antigen which acts as a vaccine, a recombinant nucleic acid, a nanoparticle, a therapeutic drug substance, a chemotherapy drug substance, an immunotherapy drug substance, or a gene therapy drug substance.

In some embodiments, the one or more transported molecules results in the biological manufacturing of at least one of: a virus-like particle (VLP), a protein-based therapeutic substance comprising at least one of a protein, a peptide, an antibody, or an enzyme, a nucleic acid-based drug substance comprising at least one of DNA, a plasmid, an oligonucleotide, an aptamer, a DNAzyme, an RNA aptamer, an RNA decoy, a microRNA fragment, or a small interfering RNA fragment, or a gene therapy drug substance.

According to some aspects of the present disclosure, a device for transiently enhancing transport of one or more target molecules across a cell membrane using electrical pulses comprises one or more electrodes configured to be positioned near a target area containing target tissue cells to be permeabilized, a power supply configured to generate and deliver electrical pulses to the one or more electrodes, and a controller operatively coupled to the power supply and configured to control the power supply to output one or more bursts of the electrical pulses, each of the bursts comprising one or more electrical pulses at a frequency rate of 50 kHz or higher and configured to permeabilize target tissue cells by non-thermal reversible electroporation, and each electrical pulse having a pulse width of 10 microseconds or less so as to permeabilize target tissue cells by non-thermal reversible electroporation, wherein the controller comprises programming for independent selection of width and amplitude of positive and negative pulses within one or more of the bursts and to allow for a delay between each burst of pulses, and wherein the controller comprises programming to control the power supply to output the pulses with an amplitude of between 250-6,000 V/cm.

According to some aspects of the present disclosure, a device for permeabilizing tissue cells of a living mammal by high frequency non-thermal reversible electroporation without using general anesthesia comprises one or more electrodes configured to be positioned near a target area containing target tissue cells to be permeabilized, a power supply configured to generate and deliver electrical pulses to the electrodes, and a controller operatively coupled to the power supply and configured to control the power supply to output one or more bursts of the electrical pulses, each burst containing multiple electrical pulses at a high frequency rate of at least 50 kHz and configured to permeabilize the target tissue cells by non-thermal reversible electroporation, and each electrical pulse having a pulse duration of 10 microseconds or less to minimize tissue stimulation, wherein the controller comprises programming to allow for independent selection of width and amplitude of positive and negative pulses within one or more of the bursts and to allow for a delay between each of the positive and negative pulses within one or more of the bursts.

According to some aspects of the present disclosure, a device for permeabilizing tissue cells by non-thermal reversible electroporation comprises two or more electrodes configured to be positioned near a target area containing target tissue cells to be permeabilized, a power supply configured to generate and deliver electrical pulses to the electrodes, a controller operatively coupled to the power supply and configured to control the power supply to output one or more bursts of the electrical pulses which include a frequency rate of 50 kHz or higher and are configured to permeabilize the target tissue cells by non-thermal reversible electroporation, a positive and a negative polarity switch in communication with the power supply, and a capacitor bank disposed between the power supply and the positive and negative polarity switches.

According to some aspects of the present disclosure, a device for permeabilizing target tissue by non-thermal reversible electroporation comprises two or more electrodes configured to be positioned near a target area containing target tissue to be permeabilized, a power supply configured to generate and deliver electrical pulses to the electrodes, and a controller in operable connection with and comprising programming of predetermined pulse parameters configured to control the power supply to output one or more bursts of the electrical pulses which include a frequency rate of 50 kHz or higher and are capable of permeabilizing the target tissue by non-thermal reversible electroporation, each burst containing multiple electrical pulses at a high frequency rate, and each electrical pulse having a pulse duration of 10 microseconds or less to minimize tissue stimulation, wherein the controller comprises programming to control a delay between the pulse bursts, and wherein the pulse parameters are sufficient to subject the target tissue to non-thermal reversible electroporation without causing muscle cells located in or surrounding the target tissue to contract.

In some embodiments, the controller comprises programming to control the power supply to generate at least one burst of bipolar electrical pulses with multiple pulses in a single phase before a polarity switch.

In some embodiments, the controller comprises programming to control the power supply to generate at least one burst of bipolar electrical pulses with multiple pulses with alternating inverted and non-inverted polarity switches.

According to some aspects of the present disclosure, a method of permeabilizing target tissue cells in vivo to locally enhance chemotherapeutic molecule uptake within tumors is provided. A series of bipolar electrical pulses may be delivered to one or more electrodes coupled with target tissue cells.

In some embodiments, the method comprises implanting at least one device for emitting electric pulses into or adjacent to the target tissue cells and delivering one or more electric pulse(s) to the target site, such that amplitude and duration of the pulse are in the range of about 1500 V/cm for 10 microseconds or less which is capable of inducing reversible electroporation.

In some embodiments, the method comprises non-invasively placing at least one device for emitting electric pulses on or around a region of the body containing the target tissue cells within and delivering one or more electric pulse(s) to the target site, such that amplitude and duration of the pulse are in the range of about 1500 V/cm for 10 microseconds or less which is capable of inducing reversible electroporation.

In some embodiments, multiple pulses are administered in a pulse burst having a duration of less than 10 ms.

In some embodiments, multiple pulses are administered in a pulse burst having an amplitude of about 500V/cm to 1500 V/cm.

In some embodiments, the pulse duration is up to 10 microseconds.

In some embodiments, a bipolar burst of pulses is delivered.

In some embodiments, a bipolar burst of pulses is delivered with multiple pulses in a single phase before a polarity switch.

In some embodiments, a bipolar burst of pulses is delivered with multiple pulses with alternating inverted and non-inverted polarity switches.

According to some aspects of the present disclosure, a method of delivering electric pulses such that amplitude and duration of a single polarity are selected to be capable of administering electroporation to electrically excitable tissue with reduced stimulation of the tissue as compared with higher amplitude and longer duration pulse protocols is provided.

In some embodiments, the electrical pulses comprise an electric waveform, and waveform is at least one of a rectangular waveform, a ramp waveform, a decaying exponential waveform, a sinusoidal waveform, a triangular waveform, or a step pulse waveform, and the waveform is unipolar or bipolar.

According to some aspects of the present disclosure, a device for transiently enhancing transport of one or more target molecules across the cell membrane using electrical pulses comprises one or more electrodes adapted to be positioned near cells to be permeabilized, a power supply adapted to generate and deliver electrical pulses to the electrodes, and a controller in operable connection with and comprising programming of predetermined pulse parameters configured to control the power supply to output one or more bursts of the electrical pulses, each burst containing one or more electrical pulses at a frequency rate of 0.1 Hz or higher and capable of permeabilizing cells, and each electrical pulse having a pulse width of 10 microseconds or less, wherein the controller comprises programming for independent selection of width and amplitude of positive and negative pulses within one or more of the bursts and to allow for a delay between each burst of pulses, and wherein the controller comprises programming to control the power supply to output the pulses with an amplitude of between 100-15,000 V/cm.

According to some aspects of the present disclosure, a device for permeabilizing target tissue is provided, wherein the controller comprises programming to control a delay between the pulse bursts, and wherein the pulse parameters are sufficient to permeabilize the cells without causing muscle cells located in or surrounding the target tissue to contract.

In some embodiments, the controller comprises programming to control the power supply to generate at least one burst of bipolar electrical pulses with multiple pulses in a single phase before a polarity switch.

In some embodiments, the controller comprises programming to control the power supply to generate at least one burst of bipolar electrical pulses with multiple pulses with alternating inverted and non-inverted polarity switches.

According to some aspects of the present disclosure, a method of permeabilizing cells in vitro to administer a large molecule for biological manufacturing of biomolecules is provided, wherein a series of bipolar electrical pulses are delivered to one or more electrodes coupled with target cells in suspension.

In some embodiments, cells are continuously infused through a reaction chamber containing electrodes.

In some embodiments, the electrical dose administered is controlled by the flow rate through the reaction chamber.

In some embodiments, reagents and cells are isolated and mixed together prior to entering the reaction chamber.

In some embodiments, cells and reagents are combined in a vessel prior to being infusion through the reaction chamber.

Aspects of the present disclosure are not limited to the above. Other devices, apparatuses, systems, and/or methods according to example embodiments will become more apparent to one of ordinary skill in the art upon review of the following drawings and detailed description. It is intended that all such additional embodiments, in addition to any and all combinations of the above embodiments, be included within this description, be within the scope of the present disclosure, and be protected by the accompanying claims.

### BRIEF DESCRIPTION OF THE DRAWINGS

The accompanying drawings, which are incorporated in and constitute a part of the specification, illustrate example embodiments of the present disclosure and, together with the description, serve to explain principles of the present disclosure.

FIG. 1A is a schematic diagram illustrating a system for using a ring-and-pin electrode to deliver electrical pulses to 3D tissue models, according to some embodiments of the present disclosure.

FIG. 1B is a schematic diagram illustrating a ring-and-pin electrode within a tissue model, according to some embodiments of the present disclosure.

FIG. 2 is a schematic diagram illustrating algorithmic quantification of live and dead regions of cells, according to some embodiments of the present disclosure.

FIG. 3 is a schematic diagram illustrating domain geometries of a two-dimensional (2D) axisymmetric model of a ring-and-pin electrode inserted into gel and a resulting mesh used for electric field simulations, according to some embodiments of the present disclosure.

FIG. 4 is an example graph illustrating electric field thresholds lumped by cell type, according to some embodiments of the present disclosure.

FIGS. 5A and 5B are example graphs illustrating average calculated electric field thresholds for treatments, according to some embodiments of the present disclosure.

FIGS. 6A to 6C are example graphs illustrating lumped sum analysis of average calculated electric field thresholds filtered by dose, rate, and waveform, respectively, according to some embodiments of the present disclosure.

FIG. 7 is an example graph illustrating average temperature profiles, according to some embodiments of the present disclosure.

FIGS. 8A to 8D are schematic diagrams illustrating GFP expression in HEK293 gels for different waveforms, according to some embodiments of the present disclosure.

FIG. 9 is an example graph illustrating the number of GFP expressing cells seen in each gel, according to some embodiments of the present disclosure.

FIGS. 10A and 10B are example graphs illustrating threshold data for 0.1 ms dose in HEK293 and BJ cells, respectively, according to some embodiments of the present disclosure.

FIG. 11 is a schematic diagram illustrating representative images of fluorescent microscopy of BJ cells in 3D gels, according to some embodiments of the present disclosure.

FIG. 12 is an example graph illustrating lumped analysis of rate using HEK293 data, according to some embodiments of the present disclosure.

FIG. 13 is an example graph illustrating lumped analysis of waveform using HEK293 data, according to some embodiments of the present disclosure.

FIG. 14 is a schematic diagram illustrating pore formation during electroporation, according to some embodiments of the present disclosure.

FIGS. 15A to 15F are schematic diagrams illustrating example waveforms including electrical pulses, according to some embodiments of the present disclosure.

FIG. 16 is a schematic diagram illustrating qualitative results of gWIZ-GFP transfection, RFPy transfection, and cotransfection for different waveforms, according to some embodiments of the present disclosure.

FIG. 17 is an example graph illustrating viability of cell cultures at varying electric field strengths (V/cm), according to some embodiments of the present disclosure.

FIG. 18 is an example graph illustrating a gating process after filtering for single cells, cell viability, and gWIZ-GFP expression, according to some embodiments of the present disclosure.

FIG. 19 is an example graph illustrating average viability data for treatment groups normalized to percent viability of control, according to some embodiments of the present disclosure.

FIG. 20 is an example graph illustrating average transfection data of each treatment group, according to some embodiments of the present disclosure.

FIG. 21 is an example graph illustrating single plasmid viability and transfection results, according to some embodiments of the present disclosure.

FIG. 22 is an example graph illustrating single plasmid viability and transfection results, according to some embodiments of the present disclosure.

FIG. 23 is an example graph illustrating dual plasmid viability and transfection results, according to some embodiments of the present disclosure.

FIG. 24 is a schematic diagram illustrating transfection efficiency for electroporation processes, according to some embodiments of the present disclosure.

FIG. 25A is a schematic diagram illustrating a flow through device for biomanufacturing, according to some embodiments of the present disclosure.

FIG. 25B is a schematic diagram illustrating a demonstration of a flow through process, according to some embodiments of the present disclosure.

FIG. 26 is a schematic diagram illustrating the viability of cells processed through the flow through device without an electric field, according to some embodiments of the present disclosure.

FIGS. 27A to 27C are example graphs illustrating transfection in a flow through device and optimization of flow rates and pulse waveforms, according to some embodiments of the present disclosure.

FIGS. 28A to 28F are schematic diagrams illustrating example waveforms including electrical pulses, according to some embodiments of the present disclosure.

FIG. 29 is a schematic diagram illustrating example waveforms including electrical pulses, according to some embodiments of the present disclosure.

FIGS. 30A to 30D are schematic diagrams illustrating stages of an in vitro technique for treating cells in a 3D tissue model, according to some embodiments of the present disclosure.

FIG. 31 is a schematic block diagram illustrating a system for delivering electroporation treatment using pulse waveforms, according to some embodiments of the present disclosure.

FIG. 32A is a schematic diagram of a circuit for generating symmetric and asymmetric pulse waveforms, according to some embodiments of the present disclosure.

FIG. 32B is a schematic diagram of a capacitor bank architecture, according to some embodiments of the present disclosure.

FIGS. 33A and 33B are example graphs illustrating results of treatment parameters, according to some embodiments of the present disclosure.

FIG. 34 is an example graph illustrating an electric field in a ring-and-pin electrode, according to some embodiments of the present disclosure.

### DETAILED DESCRIPTION

Hereinafter, example embodiments of the present disclosure will be described in more detail with reference to the accompanying drawings.

As discussed above, electroporation may be a physical method of facilitating intracellular delivery of impermeable molecular components. By applying an external electric field via electrical pulses, the cell membrane may be charged to a point where nanoscale pores develop in the membrane, thereby increasing cell permeability. This may enable transmembrane transport of a variety of molecules, such as, for example, large membrane-impermeable molecules (e.g., DNA). Electroporation may be utilized in numerous areas, such as, for example, in vitro and in vivo non-viral nucleic acid delivery (transfection), as well as drug delivery (e.g., electrochemotherapy), biomanufacturing, gene therapy.

Electroporation may occur when the local electric field surpasses a critical threshold, resulting in temporary defects which permeabilize the membrane to macromolecules that are otherwise incapable of entering the cell. This process may be termed reversible electroporation (RE) due to the transient nature of cell membrane permeability and relatively high levels of cell survival and DNA expression following treatment. However, if cells are exposed to an electric field intensity which surpasses a higher, lethal threshold, a rapid decline in cellular viability may be induced by a process known as irreversible electroporation (IRE). These reversible and irreversible thresholds may be impacted by, for example, pulse width, dose, temperature, and the like.

Conventional reversible electroporation technologies may employ protocols (e.g., waveforms) with millisecond long electrical pulses that are monopolar in nature. While this technology may lead to increases in intracellular delivery of molecules such as DNA, these pulses pose several challenges. For example, monopolar millisecond pulses may lead to excessive pore formation, which may jeopardize cell integrity (structure), resulting in cell necrosis and apoptosis. This process, known as irreversible electroporation, may be a challenge for reversible electroporation applications, particularly in vivo applications, where low viability is counterproductive. Since the same phenomenon drives both reversible electroporation and irreversible electroporation, attempts to maximize cell viability may come at the expense of intracellular delivery efficiency. Additionally, monopolar millisecond pulses may induce intense, painful muscle contractions in vivo, further reducing the clinical feasibility of this approach. As such, there is a need for new electroporation protocols that may overcome these limitations.

Example embodiments of the present disclosure provide various benefits and technical solutions to the foregoing and/or other problems associated with conventional technologies. For example, embodiments of the present disclosure may provide methods, devices, and systems for using electrical pulses to permeabilize cells and tissues to increase uptake of molecular components to elicit cellular responses. Embodiments of the present disclosure may provide methods, devices, and systems for permeabilizing cells and tissues using bursts of high-frequency, bipolar electrical pulses having burst durations (i.e., burst widths) on the order of microseconds and duration of single polarities on the microsecond to nanosecond scale. These pulses may be used to enhance membrane permeability or induce transient pores to form across the cell membrane, enhancing transport of compounds containing target molecule(s). Embodiments of the present disclosure may be used to improve, for example, delivery of nucleic acid vaccines, delivery of chemotherapeutics, delivery of large molecules across the cell membrane of target cells or tissue, biomanufacturing yields, and in vivo gene therapy. Embodiments of the present disclosure may be applicable to, for example, synthetic biology (gene transfection), electrochemotherapy, electrogenetherapy, electrothermal therapy, and blood brain barrier disruption.

Embodiments of the present disclosure may provide methods, devices and systems for using shorter, bipolar pulses that avoid muscle stimulation and increase cell viability while also improving cellular uptake compared to conventional technologies. Embodiments of the present disclosure may be used to deliver a variety of compounds in vitro and in vivo, such as, for example, plasmids, DNA vaccine candidates, chemotherapies, and gene therapy substances as well as for biomanufacturing, such as, for example, the development of virus-like particles and biological molecules. For example, bipolar microsecond pulses may offer substantial promise for DNA delivery via reversible electroporation. Embodiments of the present disclosure may provide methods, devices, and systems for using a class of pulse waveforms which alter the instantaneous and long-term charge balance of the cells/tissues to maximize membrane permeability effects, maximize cellular viability, and maximize particular cell functions/gene expression following treatments.

Embodiments of the present disclosure may provide methods, devices, and systems for permeabilizing cells to large molecules involving the administering of electrical energy. Embodiments of the present disclosure may provide methods, devices, and systems for introducing molecules into cells by delivering high-frequency, bipolar electrical pulses having a burst width on the order of microseconds and duration of single polarity on the microsecond to nanosecond scale through a plurality of electrodes defining a target area. Bipolar pulses may offer a set of parameters including, for example, waveform symmetry, pulse duration, pulse frequency, and dose, which can be adjusted to affect cellular responses to pulsed electric fields where both the reversible and irreversible thresholds fall within the treatment zone. For example, bipolar pulses and their associated parameters may be optimized according to embodiments of the present disclosure to enable clinical use of reversible electroporation for DNA vaccine delivery.

Embodiments of the present disclosure may provide methods, devices, and systems for producing superior quantities of cells to commercial reversible electroporation techniques, while eliminating muscle contractions seen in vivo. Embodiments of the present disclosure may provide methods, devices, and systems for tuning reversible treatment zones to enhance drug or gene delivery to large volumes while minimizing or eliminating potential lethal effects, improving gene transfection efficiency and minimize cell killing, and enhancing drug delivery across the blood brain barrier while having a minimal effect on healthy brain tissue.

Bipolar microsecond pulses may mitigate and prevent the muscle stimulation associated with electrical pulses. For example, high-frequency irreversible electroporation (H-FIRE) is an ablation modality that may be used to selectively ablate tissue without in vivo muscle stimulation, which may be an issue that affects other irreversible electroporation technologies using longer monopolar pulses. While this reduces stimulation, H-FIRE may not be amenable to reversible electroporation applications due to its ablative nature.

Reversible Electroporation Via Algorithmically Mediated Microsecond Pulses (REVAMMP) may be a protocol, which replaces long duration monopolar pulses with bursts of alternating polarity pulses to reduce muscle stimulation that can be additionally configured to improve intracellular delivery compared to existing reversible electroporation technologies without significant losses in viability.

Bipolar pulses may be used for pre-clinical IRE ablations without inducing muscle stimulation. These pulses may also maintain a homogeneous field distribution despite the surrounding heterogeneity of the tissue environment, enabling more predictable treatment outcomes. Bipolar pulses may also offer a new set of parameters including, for example, waveform symmetry, pulse duration, pulse frequency, and dose, which may be adjusted to potentially affect cellular responses to pulsed electric fields (PEFs) where both the reversible and irreversible thresholds fall within the treatment zone. Ultrashort (e.g., 0.5-2.0 μs) bipolar pulses may resolve many issues related with traditional long duration (e.g., 100 μs-10 ms) pulses. Such issues may include pain, muscle stimulation, and cardiac complications. Embodiments of the present disclosure may provide methods, systems, and devices for optimizing bipolar pulses and their associated parameters to enable clinical use of reversible electroporation for DNA vaccine delivery.

It may be helpful to identify the thresholds for reversible and irreversible electroporation for a given pulse sequence to develop effective in vivo transfection protocols for DNA vaccine delivery, as well as the development of applicators which may expose tissues to electric fields that surpass the reversible threshold while limiting those below the irreversible threshold. For example, pulses with durations less than or equal to 2 μs may be optimized for improved electroporation-mediated delivery of a model small molecule, propidium iodide (PI), through a large parameter sweep of different waveform parameters (e.g., pulse duration, asymmetry, energy delivery rate, dose, etc.). Dose may play an important role in affecting the difference between lethal and reversible thresholds. The ability to induce stable gene transfection may then be evaluated using a subset of these protocols which may be compared to conventional electroporation protocols. For example, electroporation protocols (e.g., waveforms) according to embodiments of the present disclosure may result in a 7730% in the number of transfected cells versus conventional electroporation controls, which indicates that ultrashort pulse duration bipolar waveforms may be a promising avenue for in vivo DNA vaccination.

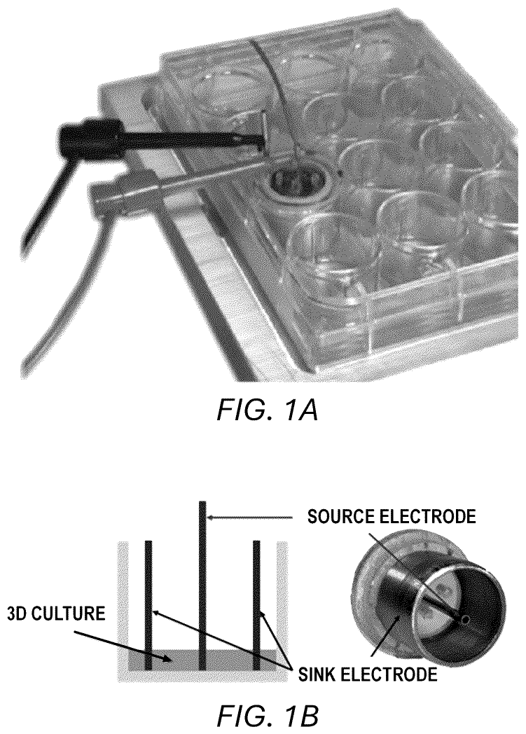

FIG. 1A is a schematic diagram illustrating a system for using a ring-and-pin electrode to deliver electrical pulses to 3D tissue models, according to some embodiments of the present disclosure. FIG. 1B is a schematic diagram illustrating a ring-and-pin electrode within a tissue model, according to some embodiments of the present disclosure.

Referring to FIGS. 1A and 1B, HEK293 and BJ cell lines may be treated individually in an established collagen-based 3D cell culture platform. Both cell lines may be cultured in Eagle's Minimum Essential Medium with 10% Fetal Bovine Serum and 1% Penicillin-Streptomycin. For all experiments, cells may be suspended in culture media at a concentration of 1×10⁶ cells/mL and combined with PurCol Easy Gel Collagen in a 1:1 ratio of cell suspension to collagen. A volume of 500 μL of this mixture (250,000 cells) may be placed in each well of a 12-well plate and allowed to incubate at 37° C. overnight to form solid three-dimensional (3D) tissue models before treatment.

A coaxial ring-and-pin electrode may be used to deliver treatments including, for example, 750 V electrical pulses with varied waveforms, doses, and delivery rates. Example treatment parameters are shown in Table 1.

TABLE 1
TREATMENT PARAMETERS

| | | | | | |
|---|---|---|---|---|---|
| Waveform (μs) | 2-1-2 | 2-1-1 | 1-1-1 | 1-1-0.5 | 0.5-1-0.5 |
| Dose (ms) | 0.1 | 1 | 10 | | |
| Delivery Rate (μs/s) | 50 | 100 | | | |

A 2-1-1 waveform may include a 2 μs positive pulse, a 1 μs delay, and a 1 μs negative pulse, but is not limited thereto. Dose may be calculated as the total energized time, which is dependent on the number of pulses delivered. Similarly, delivery rate may be quantified as the total energized time per second. A stock solution of propidium iodide (PI, 1 mg/mL) may be used as a model small molecule indicator of electroporation for optimization studies prior to the use of a DNA plasmid encoding for green fluorescent protein (GFP). Prior to treatment, each 12-well plate may be heated to physiological temperature (37° C.) before 50 μL of PI and 100 μL of media is added to each well. Each treatment may utilize one waveform, dose, and rate and may be repeated three times (N=3). During treatment, temperature may be continuously monitored via a fiber optic probe inserted into the needle electrode and a custom temperature control algorithm may be used to ensure sample temperature does not exceed 42° C. This may prevent thermal denaturing and melting of the collagen substrate which occurs at approximately 45° C.

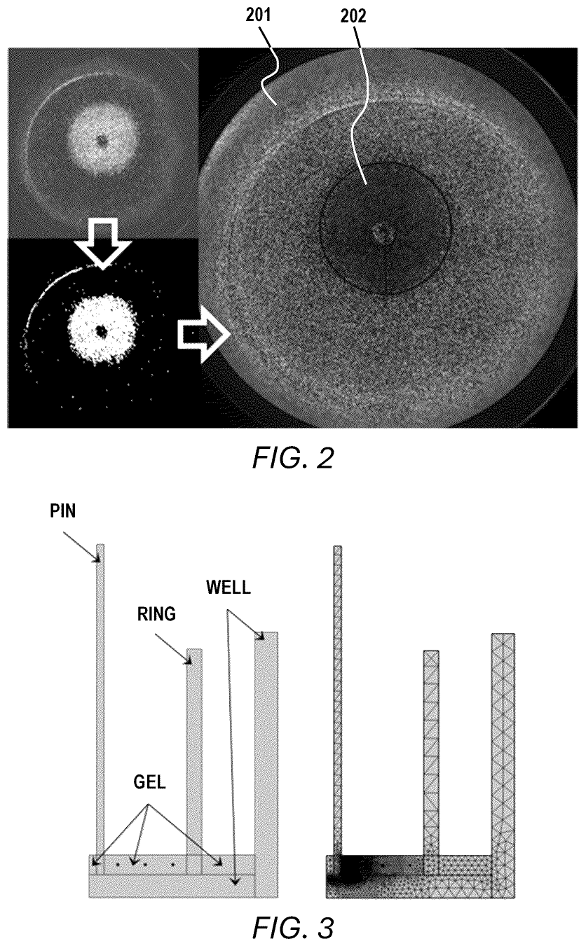

FIG. 2 is a schematic diagram illustrating algorithmic quantification of live and dead regions of cells, according to some embodiments of the present disclosure.

Referring to FIG. 2, a live region 201 and a dead region 202 are shown on the right side of FIG. 2. RGB channel variance calculations (top left of FIG. 2) may be used to binarize each image (bottom left of FIG. 2) and measure the radius of the dead region 202.

Following treatment of all wells in a plate, the tissue constructs may be washed twice with phosphate buffered saline (PBS) to remove residual PI and then imaged using fluorescent microscopy (DFC9000GT, Leica Inc., Wetzlar, Germany) to determine initial reversible electroporation thresholds. A custom MATLAB analysis algorithm may then be used to determine the radius of the resulting region to establish the reversible threshold. Samples may then be supplemented with 500 μL of fresh media and incubated for 24 hours at 37° C., at which point they may be stained and imaged to assess viability by adding Calcein AM (green/live, 2 μL of 0.4 μg/μL stock solution) and PI (red/dead, 50 μL of 1 μg/μL stock solution) to the media atop the gels and incubating for 40 minutes prior to imaging. The same image processing algorithm may then be employed to determine the radius of the dead region 202 containing non-viable cells, establishing the lethal threshold radius. Following determination of the radii associated with reversible and lethal electroporation, a COMSOL model may be used to convert these radii into electric field thresholds.

A final set of validation experiments may be conducted to demonstrate the utility of algorithmically controlled electroporation (ACE) waveforms in transfection applications. A series of preliminary experiments (data not shown) may be conducted to determine the optimal cell and DNA concentrations for these experiments with a final selection of 3×10⁶ cells/mL. Prior to treatment, 10 μL of gWIZ-GFP Plasmid (5 μg/μL, Aldeveron Inc., Fargo, North Dakota) may be added to the 3D tissue models. Samples may be incubated for 10 minutes prior to treatment using optimized protocols from the small molecule study to compare transfection outcomes to standard electrogene transfer (EGT) protocols (e.g., 8×20 ms pulses delivered at 1 Hz) using a BTX ECM 830 (Harvard Apparatus, Cambridge, Massachusetts). Samples may then be incubated overnight prior to imaging. The number of GFP expressing cells may then be quantified using a cell counting script in MATLAB.

FIG. 3 is a schematic diagram illustrating domain geometries of a two-dimensional (2D) axisymmetric model of a ring-and-pin electrode inserted into gel and a resulting mesh used for electric field simulations, according to some embodiments of the present disclosure. In particular, the 2D axisymmetric model of the ring-and-pin electrode inserted into gel is shown on the left side of FIG. 3, and the resulting mesh used for electric field simulations is shown on the right side of FIG. 3.

Referring to FIG. 3, COMSOL Multiphysics 6.0 may be used to simulate the electric field in each well. Geometric domains representing the ring-and-pin electrode and a single well of a 12-well plate may be created using caliper measurements and manufacturer datasheets. Geometries for the gel domains may be calculated using the well dimensions, assuming a 500 μL gel volume. Example dimensions of simulation geometries representing 3D tissue mimic in a single well are shown in Table 2.

TABLE 2
MODEL PARAMETERS

| Object | Dimension | Value (mm) |
|---|---|---|
| Well | Height | 17.65 |
| | Radius | 11.05 |
| | Thickness | 1.52 |
| Ring | Height | 15.00 |
| | Radius (inner) | 6.50 |
| | Radius (outer) | 7.50 |
| Pin | Height | 22.00 |
| | Radius (inner) | 0.50 |
| | Radius (outer) | 1.00 |
| Gel | Height | 1.30 |
| | Radius | 11.05 |

The Electric Currents module may be used to compute the electric field in a time-dependent 2D axisymmetric model using the following equations:

∇·J=0 (1)

J=σE (2)

E=−∇V (3)

J is the local current density, σ is the conductivity, E is the electric field, and V is the voltage.

The Heat Transfer in Solids and Electromagnetic Heating modules may be used to simulate the effects of Joule heating during treatments by solving the following equations:

∇·(−k∇T)=Q_h (4)

Q_h=R(T)J·E (5)

k is the thermal conductivity, T is the local temperature, and Qh is the electromagnetic Joule heating. Qh may be scaled by a factor R, which was equivalent to the experimental delivery rates as shown in Table 1, with unique values based on the treatment being simulated. This may reduce the computation time of simulations as individual waveforms may be represented in terms of rate rather than programming individual pulse functions.

A probe point may be placed inside of the central electrode at half of the gel height to represent the fiber-optic temperature probe used during in vitro experimentation. When the probe temperature exceeds 42° C., R (T) may be set to zero instead of the specified rate until the probe temperature returned to a value below this threshold to simulate the algorithmic temperature control used experimentally. An integration of the resulting on-time may be used to stop energy delivery after the target dose was reached. Comparison of the experimental and simulated temperature profiles may be used to confirm the validity of the COMSOL model.

A 750V boundary condition may be applied to the topmost surface of the central electrode pin:

V=750[V] (6)
and the topmost boundary of the electrode ring may be set to ground:

V=0[V] (7)
to represent the location where electrical leads were placed during the experiment. All other domain boundaries may be set as electrical insulation:

n·J=0 (8)

For the stainless-steel electrode components and polystyrene well plate, electrical conductivities may be set to 4.032×10⁶ S/m and 1×10⁻¹¹ S/m, respectively. The temperature-dependent gel conductivity (σ(T)) may be modeled using an established constitutive equation:

σ(T)=α+β·T[S/m] (9)
where α is 0.8277 S/m, β is 0.0323 S/(m·° C.), and T was the local temperature in degrees Celsius.

A 37° C. boundary condition may be applied to the base of the model to represent the heating element used to reach physiological temperature. All other domain edges which did not interface with another domain may be assigned Surface to Ambient Radiation boundaries with emissivity of 0.97, 0.9 and 0.075 depending on if the domain was part of the plate, gel, or electrode, respectively.

Two free triangular meshes may be generated with the finer mesh of the two being used on the gel domain to improve solution accuracy at the boundaries where the gel and electrode interfaced. Adaptive meshing may be utilized to further improve the solution quality in regions where the electric field was expected to change dramatically with respect to spatial variables. Three iterations of adaptive meshing may result in the final combined mesh (see FIG. 3), which includes 4,221 elements. A parametric sweep of the six combinations of energy delivery rates (50 μs/s, 100 μs/s) and doses (0.1, 1, and 10 ms) may require 3 minutes 14 seconds to solve using a ten core Intel i7-6950× processor with 64 GB of RAM.

There may be a typical exponential decay in electric field strength between the center pin and outer ring. The use of active temperature control may result in less than 1% difference in the electric field distribution at the end of each of these simulations. However, when calculating thresholds from experimental data, the electric field distribution used may be matched to the experimental parameters.

A one-tailed, paired Student's T Test may be used to determine if mean reversible thresholds were statistically different (a=0.05, p<0.05) from the lethal threshold for a single given parameter set. A two-tailed Student's T Test with equal variances may be used to determine if reversible and lethal thresholds across differing parameter sets were statistically different (a=0.05, p<0.05).

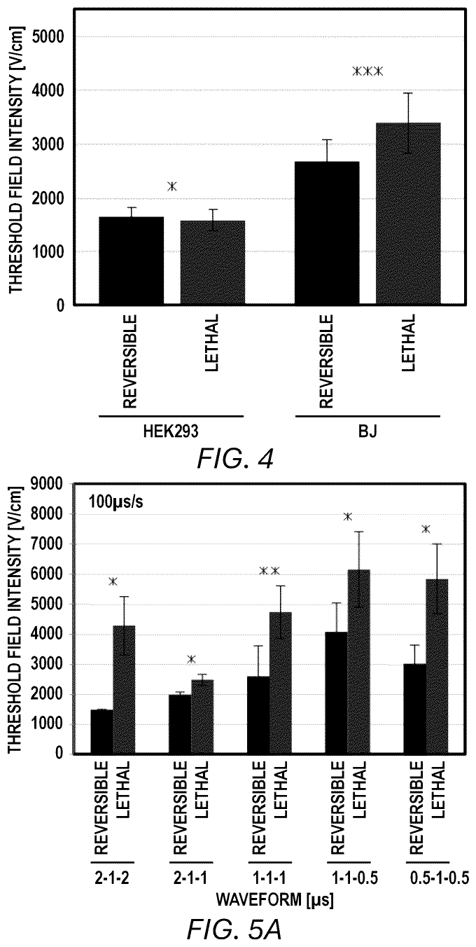

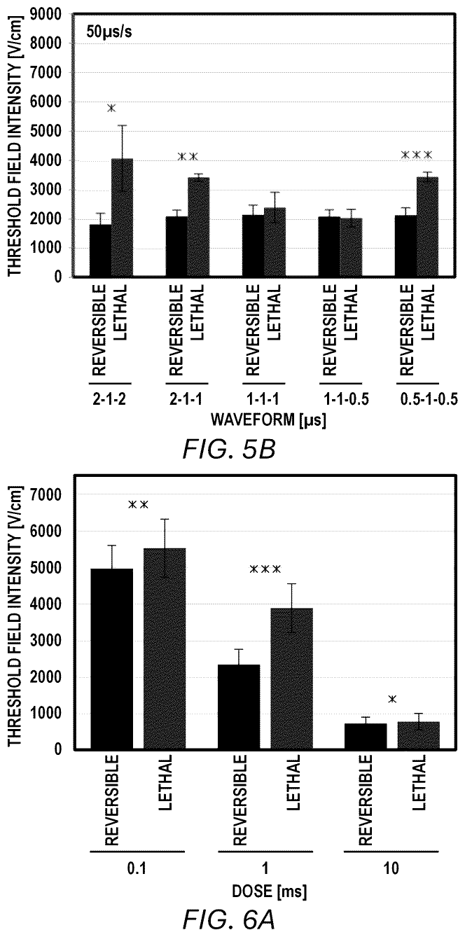

FIG. 4 is an example graph illustrating electric field thresholds lumped by cell type, according to some embodiments of the present disclosure. FIGS. 5A and 5B are example graphs illustrating average calculated electric field thresholds for treatments, according to some embodiments of the present disclosure.

Referring to FIG. 4, error bars represent one standard deviation of the mean for each threshold. In all cases, significance is denoted as *(p<0.05), **(p<0.01) and ***(p<0.001). Significant differences between reversible and lethal thresholds within the same group are denoted by asterisks directly above bars.

Referring to FIGS. 5A and 5B, average calculated electric field thresholds are shown for each treatment with a 1 ms dose in BJ cells. Higher thresholds may be seen in treatments delivered at 100 μs/s (see FIG. 5A) compared to treatments delivered at 50 μs/s (see FIG. 5B). Error bars represent one standard deviation of the mean for each threshold. In all cases significance is denoted as *(p<0.05), **(p<0.01) and ***(p<0.001). Significant differences between reversible and lethal thresholds within the same group are denoted by asterisks directly above bars.

Referring to FIGS. 4, 5A, and 5B, there may be significant differences between the reversible and lethal threshold field intensities for each cell line (p<0.001) (see FIG. 4). Because of this, each cell type may undergo separate analyses. Data within each cell line may be sorted by either waveform, dose, or rate to identify trends and features related to individual parameters. Independent of the dose administered (0.1 ms, 1 ms, 10 ms), the largest differences between reversible and lethal thresholds may be observed for BJ cells. Within the BJ cell line, treatments with a 1 ms dose may exhibit the largest differences between these thresholds (see FIGS. 5A and 5B). Treatments delivered with an initial rate of 50 μs/s (see FIG. 5A) may yield smaller differences between lethal and reversible thresholds than those initiated at 100 μs/s (see FIG. 5B). The largest difference between thresholds (2812V/cm) may be observed for 1 ms doses with the 0.5-1-0.5 waveform administered at 100 μs/s. This may result in reversible and lethal radii of 1.074±0.297 mm and 0.369±0.168 mm, respectively. These may correspond to field intensities of 3022±616 V/cm and 5834±1158 V/cm, respectively (see FIG. 5B).

The second largest difference between thresholds (2805 V/cm) and the largest ratio between thresholds (2.9:1) may be found for 1 ms doses with the 2-1-2 waveform administered at 100 μs/s (see FIG. 5B). This may result in reversible and lethal radii of 2.393±0.046 mm and 0.674±0.249 mm, respectively. These may correspond to field intensities of 1480±22.69 V/cm and 4286±970.1 V/cm, respectively (see FIG. 5B).

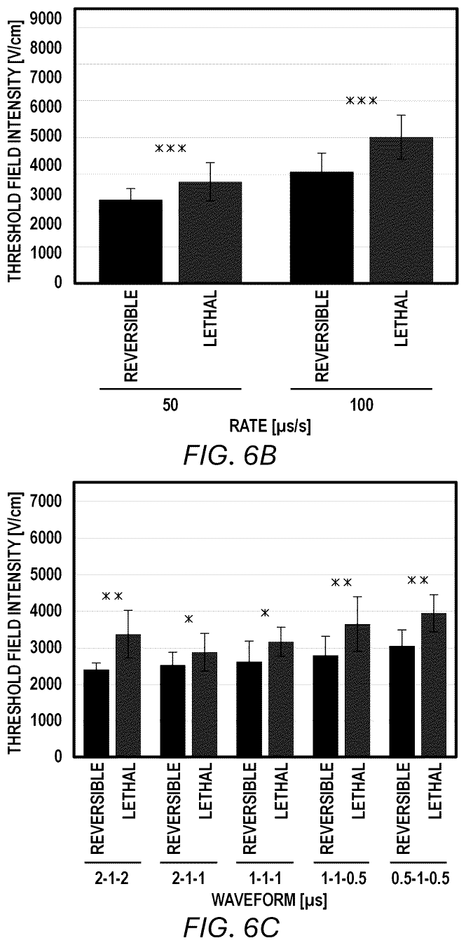

FIGS. 6A to 6C are example graphs illustrating lumped sum analysis of average calculated electric field thresholds filtered by dose, rate, and waveform, respectively, according to some embodiments of the present disclosure.

Referring to FIGS. 6A to 6C, a lumped analysis may be conducted to evaluate which parameters had the largest impact on reversible and lethal thresholds. Treatment groups may be lumped together by cell type and the parameter under investigation (dose, delivery rate, or waveform) and then averaged. Dose may have the largest impact on the magnitude of each threshold (see FIG. 6A) with a clear trend indicating that increasing dose may decrease the electric field necessary to induce reversible and lethal effects. In the lumped analysis, the largest difference between reversible and lethal thresholds may be achieved with a 1 ms dose.

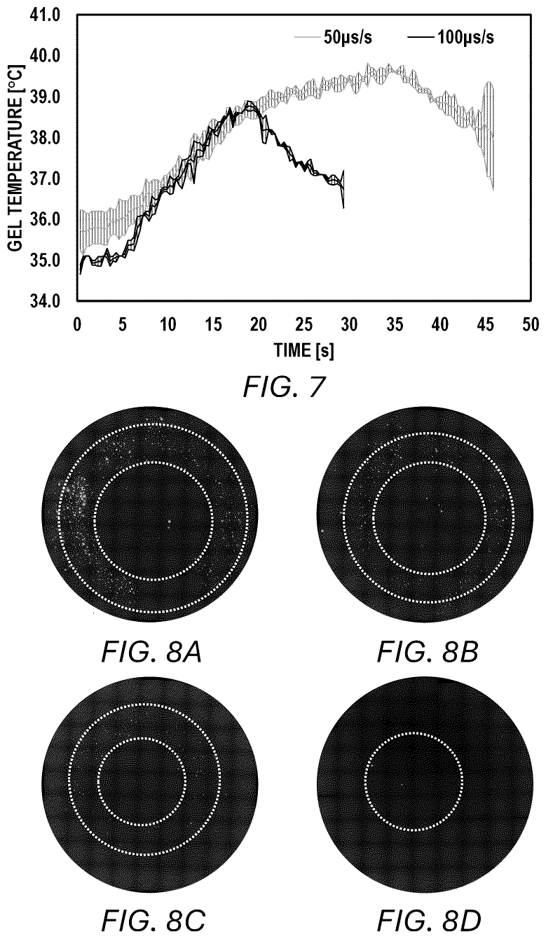

FIG. 7 is an example graph illustrating average temperature profiles, according to some embodiments of the present disclosure. In particular, FIG. 7 illustrates average temperature profiles of 50 μs/s (black) and 100 μs/s (red) treatment groups of the 2-1-2, 1 ms treatments in gels containing BJ cells. The effects of temperature control may be more obvious in longer treatments where a decrease in the rate of heating is more easily discerned.

Referring to FIGS. 6A to 6C and 7, the rate of energy delivery (see FIG. 6B) may also have a significant impact on the reversible and lethal thresholds with treatments administered with a higher initial rate having higher respective thresholds, but also a larger mean difference between reversible and lethal thresholds. These experiments may be administered with temperature control and the rate of energy delivery may be modulated to prevent heating beyond 42° C. (see FIG. 7), so these peak rates may only be maintained for a brief period at the beginning of the treatment.

When grouped by pulse waveform (see FIG. 6C), significant differences may be found between the reversible and lethal thresholds for all waveforms. However, there may not be statistically significant differences between different treatment groups. The 2-1-2 waveform may have the largest mean difference between thresholds (963V/cm). This lumped analysis indicates that a 2-1-2 waveform with a 1 ms dose administered with an initial rate of 100 μs/s may be ideal as it results in the largest safety margin between reversible and lethal effects.

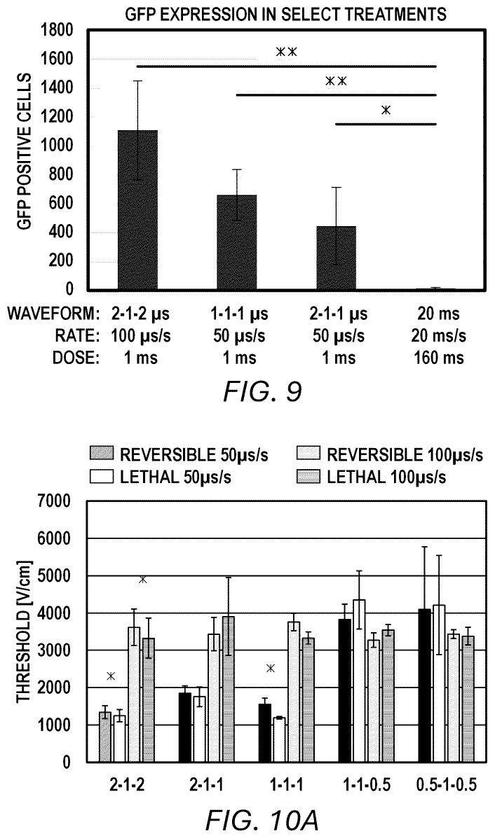

FIGS. 8A to 8D are schematic diagrams illustrating GFP expression in HEK293 gels for different waveforms, according to some embodiments of the present disclosure. In particular, FIG. 8A illustrates GFP expression in HEK293 gels for 2-1-2, 1 ms, 100 μs/s at 750 V, FIG. 8B illustrates GFP expression in HEK293 gels for 1-1-1, 1 ms, 50 μs/s at 750 V, FIG. 8C illustrates GFP expression in HEK293 gels for 2-1-1, 1 ms, 50 μs/s at 750 V, and FIG. 8D illustrates GFP expression in HEK293 gels for 8×20 ms pulses delivered at 1 Hz and at 80 V. Dotted lines in FIGS. 8A to 8D mark reversible and lethal thresholds. FIG. 9 is an example graph illustrating the number of GFP expressing cells seen in each gel, according to some embodiments of the present disclosure. In FIG. 9, error bars represent one standard deviation of the mean number of GFP positive cells for each treatment. Significant differences between the chosen treatments and a traditional electroporation protocol are denoted as *(p<0.05) and **(p<0.01).

Referring to FIGS. 8A to 8D and 9, a subset of protocols may be investigated to identify the extent to which gene transfection could be achieved with ACE waveforms. This may include the 2-1-2 waveform with a 1 ms dose administered with an initial rate of 100 μs/s treatment as well as treatments using a 1-1-1 and 2-1-1 waveform each with a 1 ms dose delivered at 50 μs/s. These may be compared to outcomes to with a standard in vivo transfection protocol (8×20 ms pulses delivered at 1 Hz). 24 hours post treatment, a torus shaped region of GFP expressing cells may be observed, indicating an external region of successful transfection and an internal region of cell death (see FIGS. 8A to 8D). The most substantial difference between field intensities, demarcated by larger transfection regions, may be seen in the 2-1-2, 1 ms, 100 μs/s treatment group (see FIG. 8A). This region may decrease in size in the 1-1-1, 1 ms, 50 μs/s group (see FIG. 8B) and then may further decrease again in the 2-1-1, 1 ms, 50 μs/s group (see FIG. 8C). The group which received 8×20 ms pulses delivered at 1 Hz had a significantly smaller transfection region (p<0.05) than any of the other treatment groups (see FIG. 8D). This trend in transfection zone mirrors the trend in the number of GFP positive cells which were found in each group (see FIG. 9). The 2-1-2, 1 ms, 100 μs/s treatment demonstrated a significant 7730% increase in GFP positive cells compared to traditional in vivo protocols (p<0.01). The 1-1-1, 1 ms, 50 μs/s group and the 2-1-1, 1 ms, 50 μs/s had 4602% (p<0.01) and 3088% (p<0.05) increases compared to the traditional protocol, respectively.

In the context of DNA vaccine delivery, there may be several factors that may need to be considered in determining an optimal treatment protocol. It may be helpful to select parameters which limit changes to traditional clinical workflows and limit pain, discomfort, and inconvenience to patients. Limiting the overall treatment time may be a concern, as prolonged administration (on the order of minutes) may hinder clinical adoption of DNA vaccines with electroporation compared to mRNA injections.

The electrode applicator (ring-and-pin electrode) used in embodiments of the present disclosure may be designed to produce a large variation in field intensities. This may enable robust quantification of thresholds based on the geometric distribution of reversibly electroporated and dead cells. As a result, protocols may be identified which produce significant volumes of reversible electroporation in the absence of any lethal effects. The use of this applicator geometry clinically may pose a challenge for in vivo DNA delivery where production of a necrotic wound would be counterproductive from efficacy and public perception perspectives. Clinical applicators may need to be carefully designed to achieve field intensities which are limited to those which only induce reversible effects and it may be necessary to select protocols in which fewer cells are successfully transfected to preserve viability in heterogeneous tissues.

FIGS. 10A and 10B are example graphs illustrating threshold data for 0.1 ms dose in HEK293 and BJ cells, respectively, according to some embodiments of the present disclosure. In the BJ cells (FIG. 10B), a large difference may be found between thresholds for the 1-1-0.5 and 0.5-1-0.5 waveforms delivered at 100 μs/s. However, these treatment zones may be quite small and may require much higher field strengths to reach. Error bars represent one standard deviation of the mean for each threshold. Significant differences between reversible and lethal thresholds within the same group are denoted by asterisks directly above the bars as *(p<0.05) and **(p<0.01).

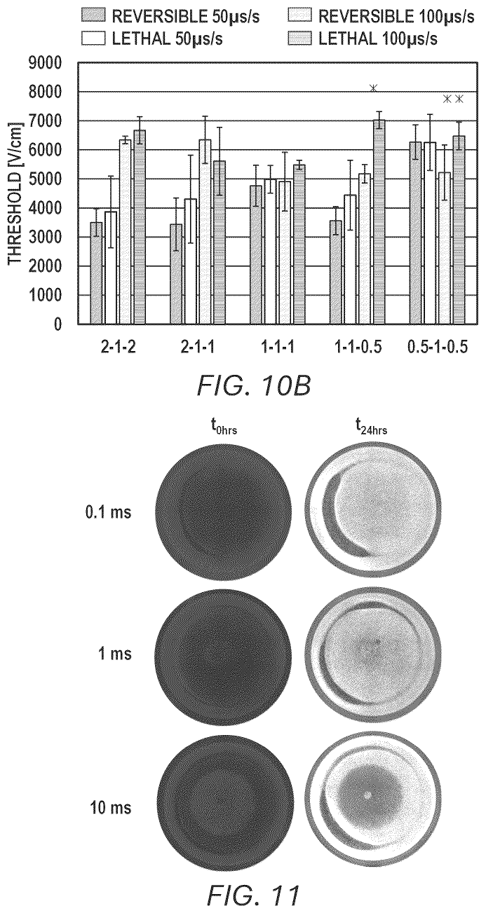

FIG. 11 is a schematic diagram illustrating representative images of fluorescent microscopy of BJ cells in 3D gels, according to some embodiments of the present disclosure. In particular, FIG. 11 illustrates representative images of fluorescent microscopy of BJ cells in 3D gels, following treatment with a 2-1-2 waveform and a delivery rate of 100 μs/s at each experimental dose. The 0.1 ms dose may have minimal uptake, but high viability. The 10 ms dose may have the largest regions of reversible electroporation, but viability may decline rapidly after 24 hours. The 1 ms dose may demonstrate larger reversible regions compared to the 0.1 ms dose while also maintaining a practical and statistically significant difference between reversible and lethal regions. Original image brightness and contrast may be increased for better visibility.

Referring to FIGS. 10A, 10B, and 11, large differences in the field intensities of 0.1 ms dose groups (see FIGS. 10A and 10B) indicate that favorable outcomes may be possible for very short doses. However, these treatments may result in very small treatment zones (1 mm), indicating little to no loss in viability, but also minimal small molecule uptake (see FIG. 11). Higher treatment voltages may be used to expand these treatment volumes. However, this may increase the potential for muscle stimulation and may increase treatment times if temperature control is necessary to prevent induction of deleterious thermal injury. It should be noted that 0.5-2.0 μs pulses have been used to treat tumors in awake veterinary patients with voltages up to 2000 V, indicating that increases beyond 750 V pulses may be feasible to increase the volume of cells transfected.

With voltage fixed, for example, to 750 V, the largest regions of reversible electroporation may be found in the 10 ms dose group. However, cell viability may rapidly decline after 24 hours (see FIG. 11), indicating that these treatments may have limited potential for gene transfection and could result in significant necrosis at the treatment site. The 1 ms dose may demonstrate larger reversible regions compared to the 0.1 ms dose while also maintaining a practical and statistically significant difference between reversible and lethal regions, suggesting that doses on the order of 1 ms are optimal for gene transfection (see FIG. 11).

Significant differences may be found for reversible and lethal thresholds between the two cell lines evaluated (see FIG. 4). Pulses on the order of one microsecond may selectively target and kill one cell type over another in 3D constructs at room temperature. Embodiments of the present disclosure indicate that differences in lethal responses may continue to persist at physiological temperatures and this selectivity may extend to reversible electroporation and gene transfection. The difference in electroporation responses may be due to variations in cell morphology and membrane kinetics. Such a phenomenon may have potential to selectively target cells of interest while sparing other cells within heterogeneous tissue, for example, targeting antigen-presenting cells in dermal tissue to increase immunogenicity of a vaccine.

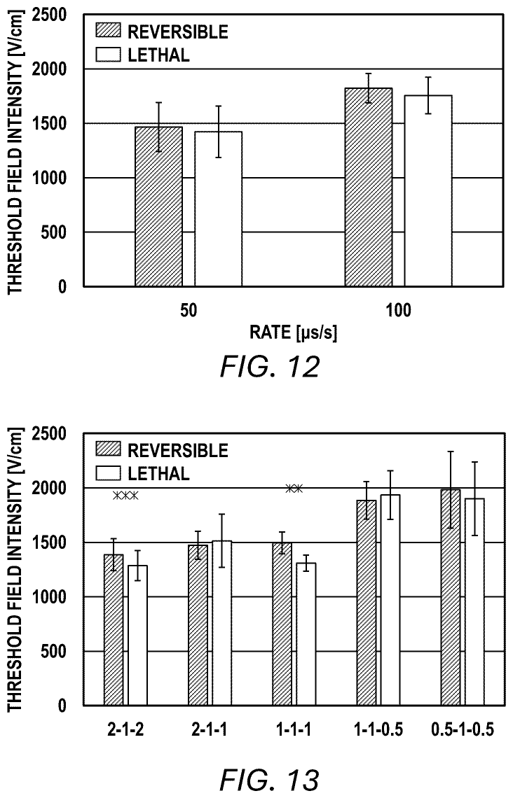

FIG. 12 is an example graph illustrating lumped analysis of rate using HEK293 data, according to some embodiments of the present disclosure. In FIG. 12, the 1-1-1 waveform may have the largest difference between thresholds. The 2-1-2 waveform may have the next largest difference, and slightly lower thresholds between these two waveforms. Error bars represent one standard deviation of the mean for each threshold. Significant differences between reversible and lethal thresholds within the same group are denoted by asterisks directly above the bars as **(p<0.01) and ***(p<0.001). FIG. 13 is an example graph illustrating lumped analysis of waveform using HEK293 data, according to some embodiments of the present disclosure. In FIG. 13, the 1-1-1 waveform may have the largest difference between thresholds. The 2-1-2 may have the next largest difference, and slightly lower thresholds between these two waveforms. Error bars represent one standard deviation of the mean for each threshold. Significant differences between reversible and lethal thresholds within the same group are denoted by asterisks directly above the bars as **(p<0.01) and ***(p<0.001).

Referring to FIGS. 12 and 13, the two cell lines may also respond differently to the rate at which pulses were delivered. For example, the lumped analysis may indicate that for HEK293 cells, the 50 μs/s rate yields a lower reversible threshold intensity and a higher lethal intensity compared to the 100 μs/s rate, although these differences may not be statistically significant (see FIG. 12). In BJ cells, the 100 μs/s rate may have a larger difference between reversible and lethal thresholds compared to 50 μs/s. Thus, it may not be possible to select a clear optimal rate for all cell lines. However, faster rates may reduce overall treatment times, which may make them more clinically viable.

Similar to rate, there may not be a clearly optimal waveform for both cell lines. The 2-1-2 waveform may result in a large difference between lethal and reversible thresholds. However, the 1-1-1 waveform may have a larger difference for the HEK293 cells (see FIG. 13). Both waveforms may have similar reversible thresholds while the symmetric 2-1-2 waveform may require a higher field strength to induce lethal effects. For this reason, the 2-1-2 waveform may be used to assess transfection efficacy.

Although the type of waveform may be found to have a substantial effect on reversible and lethal thresholds, the lumped analysis may not demonstrate any statistically significant differences between the thresholds for different waveforms. This may suggest that waveform optimization may need a closer understanding of membrane and target molecule kinetics, especially in the case of charged molecules such as DNA. Asymmetric waveforms may offer enhanced DNA uptake due to electrophoretic effects. A 2-1-1 treatment may be evaluated in the GFP validation experiment, and may demonstrate a 3088% increase in transfected cells compared to traditional electroporation.

Previous EGT studies may indicate that an electric field of approximately 200 V/cm is optimal for in vivo gene transfection. Computational models may be used to determine an experimental voltage (e.g., 80V) which achieved an approximately equivalent diameter encircled by this 200 V/cm threshold as the 1500 V/cm threshold associated with ACE reversible electroporation at 750 V.

ACE protocols according to embodiments of the present disclosure may result in orders of magnitude increases in the number of cells transfected compared to traditional EGT protocols (8×20 ms). This may be due to the existence of a significantly larger safety margin between reversible and lethal thresholds for ACE protocols. This safety margin may result in torus-shaped regions of transfection not seen with the traditional EGT protocol, indicating that ACE is capable of transfecting cells with a wider range of electric field intensities than traditional EGT approaches.

In 3D tissue models, transfection yields for the EGT protocol may be extremely. This is in contrast to experiments on cells in suspension where yields may be substantially higher (20%-60%). This may be due to a number of factors including uncontrolled thermal transients which routinely reached 50° C. despite the use of substantially lower voltages (80V) for EGT than those used in the ACE protocols (750V). This may result in some melting of the collagen structure in proximity to the center electrode pin. Therefore, it may not be possible to explore higher voltages to investigate if higher field strengths improve EGT transfection outcomes. Conversely, reducing the voltage may result in smaller volumes exposed to fields of 200 V/cm further limiting comparisons between these ACE and EGT approaches. Embodiments of the present disclosure found that ACE protocols increase the number of cells transfected by between 3088% and 7730%.

Reversible electroporation offers a promising avenue for implementation of compounds such as, for example, DNA vaccines. Embodiments of the present disclosure provide methods, devices, and systems for ultrashort bipolar waveforms, which may improve in vivo transfection efficacy while addressing challenges which have limited clinical electroporation. Embodiments of the present disclosure provide methods, devices and systems for using ultrashort bipolar waveforms that avoid muscle stimulation and increase cell viability while also improving cellular uptake compared to conventional technologies. Because of this, embodiments of the present disclosure may be used to deliver a variety of compounds in vitro and in vivo, such as, for example, plasmids, DNA vaccine candidates, chemotherapies, and gene therapy substances as well as for biomanufacturing, such as, for example, the development of virus-like particles and biological molecules.

Interest in genetic payload delivery has received increasing attention with recent enhancements in drug delivery, DNA vaccines, and CRISPR therapies. For example, electroporation may be one non-viral/physical method of delivery. Electroporation may utilize pulse electric fields to create nanoscale-level pores through which payloads can be delivered (i.e., transfected) into the cell. Fine tuning of electroporation protocols may be important, as proliferation of such pores can lead to necrosis and later apoptosis. Monopolar electroporation protocols may either reduce cell viability while transfecting much of the cell population or maintain high cell viability while performing poorly on transfection. Bipolar pulse waveforms with different pulse patterns may be utilized to enhance viability, single plasmid transfection, and dual plasmid cotransfection. For example, bipolar pulse waveforms may increase single plasmid and dual plasmid transfection as compared to monopolar pulse waveforms, without impacting cell viability.

As discussed above, electroporation is a physical method of facilitating intracellular delivery of impermeable molecular components. Electroporation may increase cell permeability by delivering electrical pulses to the cell and may be utilized in numerous areas including, for example, tumor ablation, drug delivery, and gene delivery. The electrical pulses may create nanoscale-level pores in the cell membrane that enable transmembrane transport of large membrane impermeable molecules. Delivery of these components (e.g., through the nanoscale-level pores) may be referred to as transfection. For example, target molecules may be administered to an environment surrounding target cells or tissue, and electrical pulses may be delivered to the target cells or tissue to create nanoscale-level pores in the cell membrane(s) of the target cells or tissue that enable transmembrane transport of the target molecules.

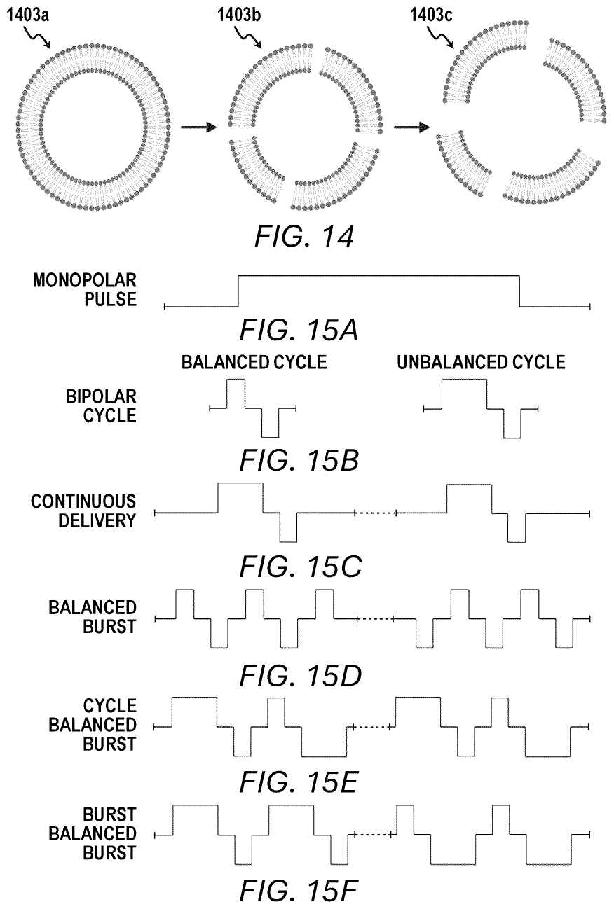

FIG. 14 is a schematic diagram illustrating pore formation during an electroporation process, according to some embodiments of the present disclosure.

Referring to FIG. 14, an untreated cell 1403a may subjected to electroporation. A challenge with electroporation may be that excessive pore formation jeopardizes cell integrity (structure), resulting in cell necrosis and apoptosis. This type of electroporation, which may be referred to as irreversible electroporation 1403c, may be tuned to specifically kill cells (e.g., for oncology applications). However, it may be challenging for in vivo genetic engineering applications where production of a necrotic wound could be counterproductive. For in vivo transfection, it may be helpful to tune application protocols to favor reversible electroporation 1403b, where sufficient pores for transfection are induced without reducing viability.

The electrical pulses delivered for electroporation may range from nanoseconds (ns) to milliseconds (ms) in duration. Millisecond duration electrical pulses may induce, painful intense muscle stimulation. Recommended protocols for traditional reversible electroporation may employ the use of millisecond long pulses. Traditional monopolar electroporation hardware (MEH) may be configured to deliver an array of outputs which range in duration (e.g., 100 μs to 20 ms), with typical in vivo gene editing protocols administering eight 20 ms pulses with a 1 second delay between pulses (1 Hertz (Hz)). As mentioned above, these pulse durations may result in intense, painful muscle stimulation in vivo, which may reduce the clinical viability of the MEH approach. There may be several factors such as pulse duration, polarity, and waveform shape that affect electroporation efficiency while preserving cell viability and reducing muscle simulation. Nanosecond duration pulses may alleviate these effects, but they may require electric field strengths of 10-60 kV/cm with limited transfection efficiency, which may lead to some challenges in developing compact clinical instrumentation with these approaches. Recent investigation into microsecond and nanosecond duration pulses have been shown to facilitate in vitro genetic engineering. Such nanosecond protocols may require a relatively high electric field strength (e.g., up to 7000 V/cm) which may limit transfection volumes in vivo. To overcome such restrictions, alternating polarity pulses on the order of 1 μs may be used. Such a pattern may expand the safety margin between lethal and reversible effects and may achieve reversible electroporation with significantly lower field strengths (e.g., 750-2000 V/cm) than nanosecond waveforms.

Example embodiments of the present disclosure provide methods, devices, and systems for generating alternating polarity waveforms including intermediate duration microsecond pulses that aim to address the current clinical challenges discussed above.

FIGS. 15A to 15F are schematic diagrams illustrating example waveforms including electrical pulses, according to some embodiments of the present disclosure.

Referring to FIGS. 15A to 15F, the use of alternating polarity pulses may lead to a substantial number of possible and customizable waveforms. A bipolar waveform, for example, may have positive effects on viability, transfection, and simultaneous dual plasmid transfection.

As shown in FIG. 15A, some electroporation protocols may administer a relatively long monopolar electrical pulse at a fixed repetition rate (e.g., 20 ms pulse administered at 1 Hz). Electrical charge carriers (e.g., electrons) may flow in a single direction. This may result in a consistent electric field for the duration of the pulse leading to charge imbalances, with ions in and around the cell driven by electrokinetic forces.

Alternating polarity microsecond duration waveforms may provide benefits during an electroporation process that are not offered by a monopolar electrical pulse. As shown in FIG. 15B, an alternating polarity microsecond duration waveform may be classified as charge balanced or unbalanced. As used herein, the notation P-D-N may be used to describe the positive pulse (or positive phase) (P), inter-pulse (or interphase) delay (D), and negative pulse (or negative phase) (N) duration in microseconds (μs). Further, as used herein, a biphasic pulse including a positive pulse and a negative pulse separated by a delay (i.e., a pulse free delay) may also be referred to as an electrical pulse including first and second phases (i.e., positive and negative phases) separated by an interphase delay, where the first and second phases have opposite polarities. In other words, positive and negative pulses included in a biphasic pulse may also be referred to as first and second phases of an electrical pulse. In some embodiments, the first and second phases (i.e., the positive and negative pulses) of a biphasic pulse may be separated by an interphase delay. Further, in some embodiments, successive biphasic pulses in an electrical waveform may be separated by an interpulse delay.

As shown in FIG. 15C, in continuous delivery waveforms (i.e., protocols), the singular cycles (e.g., each including a positive pulse, a delay, and a negative pulse) that make up waveforms may be dispersed evenly over an entire treatment duration at a rate of, for example, 25-50 Hz.

As shown in FIG. 15D, in balanced burst waveforms, the cycle (e.g., the P-D-N waveform) may be repeated rapidly in succession to create a burst which is energized (e.g., for approximately 100 μs). This burst may then be repeated, for example, once or twice per second (e.g., 1 or 2 Hz) to mimic traditional electroporation protocols. In some embodiments, the delay may be 1 microsecond, but is not limited thereto.

The burst and continuous waveform pulses may be energized on average, for example, 200 μs out of every second to achieve a delivery rate of 200 μs/s. These burst waveforms may be configured as either symmetric (e.g., 1-1-1) or asymmetric (e.g., 2-1-1). In other words, a positive pulse and a negative pulse may have a same duration or may have a different duration.

As shown in FIG. 15E, in cycle balanced burst waveforms, the duration of the first positive pulse and the duration of the first negative pulse may be alternated. This pattern may continue so that charge balance is achieved after every two cycles. In some embodiments, the positive (P) and negative (N) durations may be alternated in successive repetitions to create a charge neutral burst of pulses energized, for example, for 100 μs (e.g., P-D-N-D-N-D-P-D . . . ). In this way, charge balance may be immediately achieved by the next bipolar cycle as the burst is being delivered. An asymmetric waveform that results in a net charge balance by an end of delivery may also be referred to as an asymmetric charge-balanced electrical waveform. For example, an asymmetric charge-balanced electrical waveform according to some embodiments herein may include positive and negative phases (i.e., positive and negative pulses), with at least some of the positive and negative phases having different durations and/or magnitudes but resulting in a net charge balance by the time the waveform is finished being delivered (i.e., by an end of delivery). In other words, the positive and negative phases may be transiently asymmetric (i.e., may have different durations and/or magnitudes during at least part of the delivery of the electrical waveform) but result in a net charge balance by the time the electrical waveform is finished being delivered. The waveform shown in FIG. 15E may also be referred to as an asymmetric charge-balanced electrical waveform.

As shown in FIG. 15F, in burst balanced burst waveforms, the polarity and duration of each cycle may remain the same for the first half of the burst. Once half of the cycles have been delivered, the duration of the positive pulses and the duration of the negative pulses in the first half of the burst may be inverted so that the cycles in the second half of the burst are opposite in polarity to the cycles in the first half of the burst. For example, the positive (P) and negative (N) durations may be switched halfway through the burst, where the negative (N) duration of the first half of the pulses becomes the positive (P) duration of the second half of the pulses, and the positive (P) duration of the first half of the pulses becomes the negative (N) duration of the second half of the pulses (e.g., P-D-N-D-P-D-N-D . . . . N-D-P-D-N-D-P). In this way, unlike the balanced burst, continuous delivery, and cycle balanced burst waveforms, charge balance may not be achieved until the whole burst is finish. The waveform shown in FIG. 15F may also be referred to as an asymmetric charge-balanced electrical waveform.

In some embodiments, a continuous 2-1-2 waveform (e.g., positive pulse and negative pulse each have a duration twice as long as a delay therebetween) may be selected as a baseline waveform due to its ability to be administered in vivo with voltages up to, for example, 2000 V without the need for neuromuscular paralytics or general anesthesia. As production of a necrotic wound in vivo may be counterproductive, a voltage escalation may be performed to identify a baseline protocol capable of maintaining greater than 80% viability. Testing at these 2-1-2 settings and voltages that maintained high viabilities may ensure that the waveforms are both incapable of causing muscular contractions while maintaining cell structure and function.

To evaluate viability, HEK293 cells may be cultured in Eagle's Minimum Essential Medium (EMEM) with 10% Fetal Bovine Serum (FBS) and 1% Penicillin-Streptomycin. Cells may then be suspended in culture media at a concentration of 2×10⁶ cells/mL before being combined 1:1 with low conductivity isotonic dielectrophoresis (DEP) buffer (8.5% sucrose [wt/vol], 0.3% glucose [wt/vol], and 0.725% [vol/vol] RPMI)). 400 μL volumes of this mixture (400,000 cells) may be placed in 4 mm cuvettes and treated in a BTX Safety Stand (Harvard Apparatus) using a custom pulse generator to deliver treatments consisting of a 2-1-2 waveform, a 1 ms dose, and a 100 μs/s delivery rate, with field intensities ranging from 750 V/cm to 3000 V/cm. Treatments at each field intensity may be replicated in triplicate (N=3).

One and two hours after treatment, the contents of each cuvette may be gently mixed to resuspend any settled cells before 18 μL samples are extracted and mixed with 2 μL of Acridine Orange/Propidium Iodide (Invitrogen) to analyze viability using a LUNA (LUNA FL Dual Fluorescence Cell Counter). Samples may be analyzed according to the manufacturer's protocol to assess changes in overall viability. 1500 V/cm may be identified as the highest field strength for which viability greater than 80% was achieved and thus used for transfection experiments with more complex waveforms.

Samples may be prepared as described above with 20 μg of gWIZ-GFP Plasmid (5 μg/μL, Aldeveron Inc., Fargo, North Dakota) added to each cuvette immediately prior to treatment. Cuvettes may then receive one of the example treatments shown in Table 3, with electric field strength, dose, and delivery rate fixed to, for example, 1500 V/cm, 0.005 s, and 200 μs/s, respectively. Additionally, two positive controls (MEH, 8×20 ms pulses at 1 Hz), and a negative control (plasmid only, no electroporation) may be evaluated. Treatments may be performed in triplicate (N=3). Table 3 shows bipolar pulse waveform protocols chosen for single plasmid transfection experiments.

TABLE 3

| Positive | | Negative | Treatments |
|---|---|---|---|
| Pulse | Delay | Pulse | Administered |
| 1 | 1 | 0.5 | Continuous, |
| | | | Burst Balance, |
| | | | Cycle Balance |
| 1 | 1 | 1 | Continuous, |
| | | | Burst |
| 2 | 1 | 1 | Continuous, |
| | | | Burst Balance, |
| | | | Cycle Balance |
| 2 | 1 | 2 | Continuous, |
| | | | Burst |

After treatment, the contents of each cuvette may be aliquoted into 6-well plates containing 500 μL of 25% FBS supplemented media. Cuvettes may be carefully flushed with 500 μL of additional supplemented media to collect any remaining cells in the cuvette. Plates may be gently swirled to ensure uniform seeding and then incubated at 37° C. for 24 hours.

To analyze viability and transfection efficiency, adherent cells and those in the supernatant may be collected. Non-adherent cells may be harvested by collecting each well's supernatant. Following trypsinization of the well to collect adherent cells, the supernatant may then be added back to neutralize the trypsin. Cells may then be stained with Ghost Dye 780 (Cytek Biosciences) and fixed (BD Biosciences) prior GFP expression and viability analysis via flow cytometry.

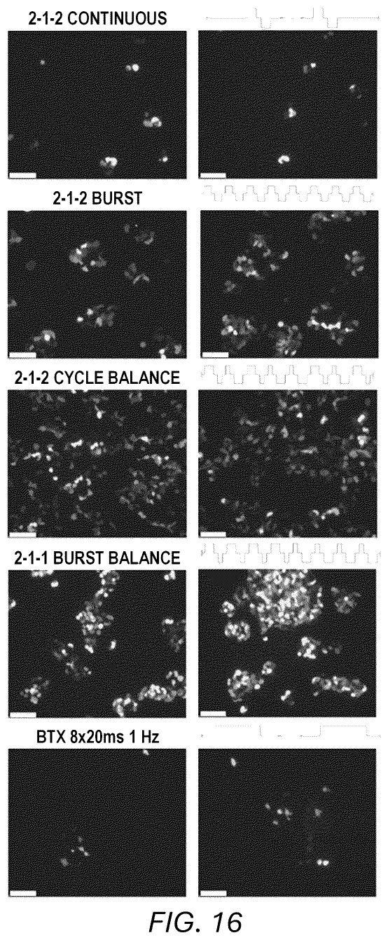

FIG. 16 is a schematic diagram illustrating qualitative results of gWIZ-GFP transfection, RFPy transfection, and cotransfection for different waveforms, according to some embodiments of the present disclosure. In particular, FIG. 16 illustrates qualitative results of gWIZ-GFP transfection (in green), RFPy transfection (in red), and cotransfection (in yellow) for different waveforms 36 hours post treatment. White scale bars are equivalent to 100 μm.

Referring to FIG. 16, simultaneous transfection of multiple plasmids may be examined. To evaluate the pattern of cotransfection in MEH protocols versus bipolar pulse waveforms, qualitative assessments of plasmid uptake may be analyzed via florescent microscopy. Four pulse waveforms may be chosen to be re-evaluated along with the BTX/MEH control. Samples maybe prepared as described previously with 20 μg of gWIZ-GFP Plasmid (5 μg/μL, Aldeveron Inc., Fargo, North Dakota) and 20 μg of tdTomato RFP (9.1 μg/μL gifted by Driss Elhanafi) added to each cuvette immediately prior to treatment. Contents of the treated cuvettes may be transferred to wells with 1.1 mL Eagle's Minimum Essential Medium (EMEM) with 25% Fetal Bovine Serum (FBS) and 1% Penicillin-Streptomycin. After 24 hours, the wells may be imaged using via florescent microscopy using a Lycia DMi8 Inverted microscope.

To implement more controls in plasmid uptake analysis, pulse waveforms may be chosen to be re-evaluated using a GFP (green fluorescent protein) plasmid and a RFP (red fluorescent protein) plasmid that were synthesized from the same plasmid backbone (GFPx and RFPx). In addition to waveforms described above, a positive control (MEH protocol), a negative control (plasmid only sham), and a lipidic transfection protocol may be evaluated.

These treatments may be administered to a cell suspension with GFPx plasmid and RFPx plasmid. Transfection efficiency with these plasmids may be compared to gWIZ-GFP. Treatments with 20 μg of the GFPx (2.75 μg/μL) or RFPx (2.75 μg/μL) were independently tested with example protocols shown in Table 4. Table 4 shows a subset of protocols chosen for cotransfection experiments. For example, all protocols may be conducted with a field strength of 1500 V/cm, dose of 0.005 s, and rate of 200 μs/s.

TABLE 4

| Positive | | Negative | Treatments |
|---|---|---|---|
| Pulse | Delay | Pulse | Administered |
| 2 | 1 | 2 | Continuous, |
| | | | Burst |
| 2 | 1 | 1 | Burst Balance, |
| | | | Cycle Balance |

To investigate the extent to which protocols enabled simultaneous co-transfection, 10 μg of the GFPx (2.75 μg/μL) and 10 μg RFPx (2.75 μg/μL) may be added simultaneously to each cuvette. Treatments may be replicated in triplicate and performed twice (N=6). The order of plasmid experiments may be randomized. Following treatment, samples may be harvested, stained, and fixed for flow cytometry as above.

Plasmid GFPx expresses the protein eGFP under the mammalian promoter hCMV. GFPx may be generated by cloning the DNA sequence coding for the green fluorescence gene eGFP on the mammalian expression vector phCMV.3 (#P003300, Genlantis, San Diego, CA, USA). The eGFP gene may be amplified with PCR using the plasmid pEGFP-N1 (Clontech Laboratories, Inc. GenBank Accession #U55762) vector containing eGFP gene DNA as a template, and the primers eGFP-F41: GTATA CTCGAG GCCACCATGGTGAGCAAG GGCGAGGAG and eGFP-R41: GAATCACTCGAGTTACTTGTACAGCTCGTCCATG. XhoI restriction sites may be added at the 5′ end of the primers for cloning purposes (underlined sequences). The PCR product may be digested with XhoI restriction enzyme and ligated to the plasmid phCMV3 linearized with XhoI restriction enzyme, using the T4 DNA ligase. The eGFP gene in GFPx may have the 5′ region close to the hCMV promoter as determined by PCR (not shown).

The plasmid RFPx expresses the gene tdTomato under the mammalian promoter hCMV. RFPx may be generated by cloning the DNA sequence coding for the TdTomato gene on the mammalian expression vector phCMV.3. The tdTomato gene may be amplified with PCR using the plasmid Fireworks RFP [PTC-] (AVA2515) (Addgene plasmid #85443; http://n2t.net/addgene: 85443; RRID: Addgene_85443) as a template using the primers tdTomato-F1: GTATA CTCGAG GCCACCATGGTGAGCAAGGGCG (XhoI restriction site underlined) and tdTomato-R1: GTATA GAATTC TTA CTTGTACAGCTCGTCCATGCCG (EcoRI restriction site underlined). The PCR product may be digested with XhoI and EcoRI restriction enzymes and ligated to the vector phCMV3 digested with the same enzymes, using the T4 DNA ligase. All restriction enzymes and T4 DNA ligase may be from New England Biolabs, Ipswich, MA, USA.

A CytoFLEX Flow Cytometer (Beckman Coulter Life Sciences) may be used to analyze plasmid uptake and viability. Compensation controls may include an unstained sample, a non-plasmid sample, a heat-treated sample which received viability staining, and samples containing one of the plasmids but were not stained for viability. The heat-treated sample may be created by heating a well plate containing untreated cells to 70° C. for 60 seconds before incubating at −4° C. for five minutes prior to harvesting via trypsin. Flow cytometry controls may be created by preparing cuvettes with either 20 μg of the GFPx plasmid, 20 μg of the RFP plasmid, 10 μg of both GFPx and RFPx, or heat treatment.

Flow cytometry data may be analyzed, for example, in Flowreada.io. A compensation matrix may be generated using compensation controls and a gating process may be used to segment single cells based on viability, presence of GFPx, and presence of RFPx when relevant. The lower limit of plasmid expression for the GFPx and RFPx expression gating may be selected to be approximately halfway between 104 and 105 [RFU]. The amount (in percent) of living cells that are expressing a plasmid out of all living cells analyzed may be referred to as the transfection efficiency.

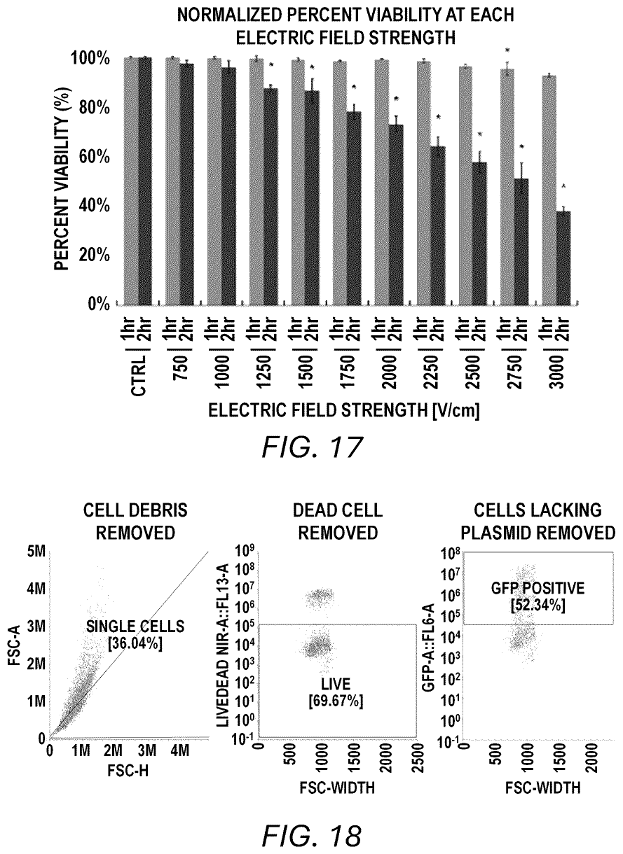

FIG. 17 is an example graph illustrating viability of cell cultures at varying electric field strengths (V/cm), according to some embodiments of the present disclosure. In FIG. 17, t is the time between treatment and fixing cells. Error bars represent standard deviation of data set. * indicates significantly different from control treatment at p<0.01.

Referring to FIG. 17, the relative change in viability may be negligible one hour after treatment for electric field strengths between 750 and 3000 V/cm. Only one treatment group, 2750 V/cm, may have a significantly lower (p<0.01) viability compared to untreated control. However, all treatment groups with a field strength of 1250 V/cm or higher may result in significantly lower viability than control when assessed two hours after treatment. For example, the 3000 V/cm treatment group may have an average viability of 93% 1-hour post-treatment which reduces to 38% 2 hours post-treatment. As the electric field strength of the treatment group increases beyond 1500 V/cm, the 2-hour viability may drop approximately 5-8% for every 250 V/cm increase in field strength until the 3000 V/cm treatment group which yields a 13% reduction in viability from the 2750 V/cm group. Two-hour viabilities of 97%, 96%, 87% and 87% may be found for the 750, 1000, 1250, and 1500 V/cm groups, respectively. Viability may fall below 80% for field strengths above 1500 V/cm. 1500 V/cm may thus be carried forward for remaining transfection experiments.

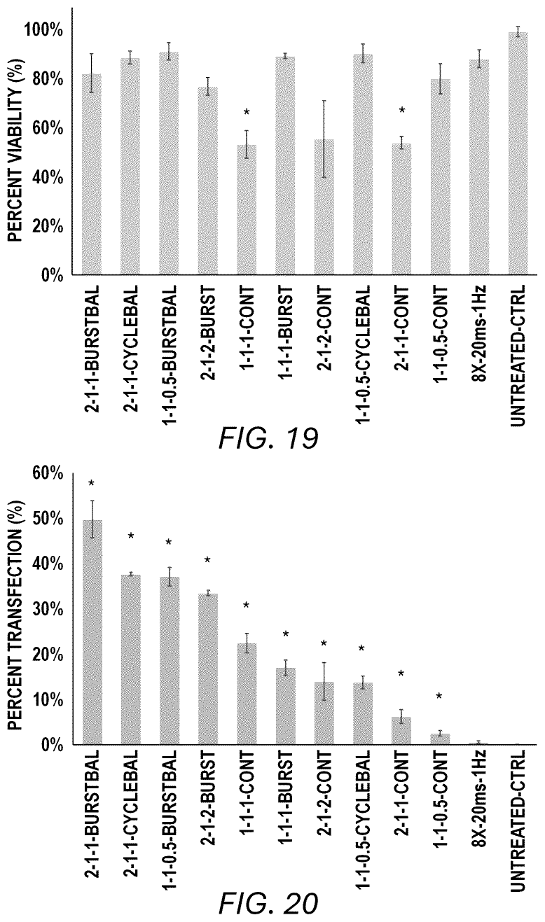

FIG. 18 is an example graph illustrating a gating process after filtering for single cells, cell viability, and gWIZ-GFP expression, according to some embodiments of the present disclosure. In particular, the single cells are shown on the left side of FIG. 18, the cell viability is shown in the center of FIG. 18, and the gWIZ-GFP expression is shown on the right side of FIG. 18. FIG. 19 is an example graph illustrating average viability data for treatment groups normalized to percent viability of control, according to some embodiments of the present disclosure. FIG. 20 is an example graph illustrating average transfection data of each treatment group, according to some embodiments of the present disclosure. Error bars represent standard deviation of data set. * indicates significantly different from current standard BTX (8×20 ms-1 Hz) treatment at p<0.01.

Referring to FIGS. 18 to 20, transfection efficiency and viability may be assessed via flow cytometry 24 hours post treatment (see FIG. 18). The 1-1-0.5 burst balance, 1-1-1 burst, and 1-1-0.5 cycle balance treatments may yield the highest relative viabilities of 92%, 90%, and 91%, respectively (see FIG. 19). The MEH protocol (8×20 ms pulses) may yield a viability of 88.89%. Only two alternating polarity treatments, 1-1-1 continuous (54%) and 2-1-1 continuous (54%), may be significantly lower (p<0.01) in viability than the MEH protocol. These two protocols may also be the only two that were significantly lower (p<0.01) in viability from the untreated control.

As shown in FIG. 20, the MEH protocol may yield 0.5% transfection of the GFP plasmid. In contrast, 50% of cells may be transfected with the 2-1-1 burst balance protocol yielding a 94× increase over the MEH protocol. The bipolar protocol (1-1-0.5 continuous) may result in 2.6% transfection, yielding a 5× increase over the MEH protocol. Transfection efficiency may be significantly greater (p<0.01) than both the untreated control and the MEH treatment for every bipolar protocol evaluated.

Referring back to FIG. 16, to assess the transfection capabilities of each treatment, two plasmids (gWIZ-GFP and RFPy) may be introduced to 5 treatment groups: 2-1-2 continuous, 2-1-2 burst, 2-1-1 cycle balance, 2-1-1 burst balance, and MEH waveforms. These specific treatment groups may be selected based on the single plasmid transfection data. In some embodiments, the 2-1-1 burst balance may be selected as the best performing waveform for transfection, and the 2-1-2 continuous treatment may be selected as the worst performing waveform for transfection (besides the MEH waveform), but the present disclosure is not limited thereto. In some embodiments, the 2-1-1 cycle balance treatment may also be selected as the second-best performing waveform for single plasmid transfection, and the 2-1-2 burst treatment may be selected as it reflects a median treatment in transfection efficiency with known clinical utility, but the present disclosure is not limited thereto.

Each group may be moved from cuvettes to well plates and imaged (e.g., on a Leica DMi8) with fixed exposure settings. Qualitatively, the 2-1-1 burst balance waveform may result in the greatest amount of cotransfection, with the 2-1-1 cycle balance, 2-1-2 burst, and 2-1-2 continuous waveforms progressively showing less evidence of cotransfection. The MEH treatment may have substantially fewer cells and lower incidence of cotransfection than the other waveforms.

Across all experiments, the three control groups may maintain similar viabilities (>95%). The 2-1-2 continuous treatment may yield the lowest viability on average with 32.61%, across all plasmid groups. The remaining waveforms may yield increasingly higher viabilities approaching those of the control groups.

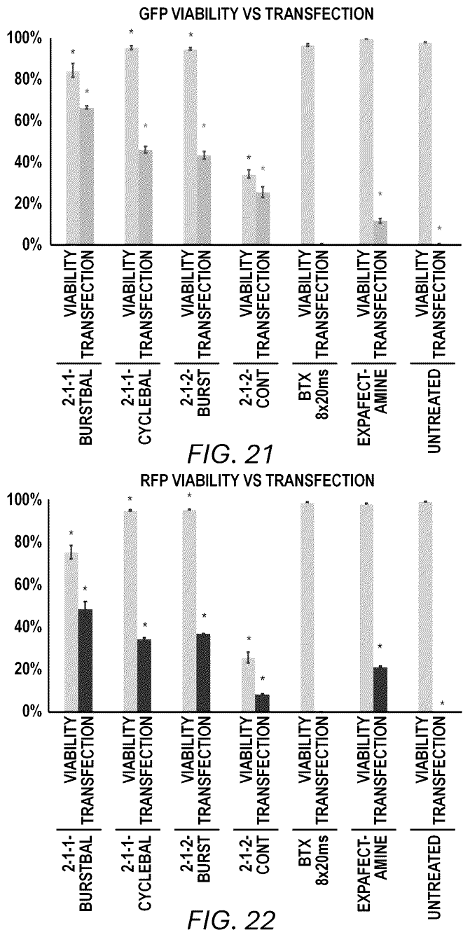

FIG. 21 is an example graph illustrating single plasmid viability and transfection results, according to some embodiments of the present disclosure. FIG. 22 is an example graph illustrating single plasmid viability and transfection results, according to some embodiments of the present disclosure. FIG. 23 is an example graph illustrating dual plasmid viability and transfection results, according to some embodiments of the present disclosure. In particular, FIG. 21 illustrates single plasmid viability and transfection results for GFPx, FIG. 22 illustrates single plasmid viability and transfection results for RFPx, and FIG. 23 illustrates dual plasmid viability and transfection results for cells exposed to GFPx and RFPx (GFPx+RFPx).

Referring to FIGS. 21 to 23, the untreated control and MEH protocol (8×20 ms pulses) may result in the least amount of transfection. For the MEH protocol, transfection efficiency may be 0.24% for the GFPx only, 0.29% RFPx only, and 0.24% for GFPx+RFPx. For the untreated control, transfection efficiency may be 0.03%, 0.02%, and 0.04% for these same respective groups. The transfection efficiency (living cells that are expressing a plasmid) may be highest for the 2-1-1 Burst Balance waveform for all plasmid experiments with a transfection efficiency of 66.40% (GFPx), 48.45% (RFPx), and 35.36% (GFPx+RFPx). The transfection efficiency of the lipidic delivery control using Expifectamine may vary depending on the plasmid and may be generally lower than all bipolar pulse waveforms for single plasmid transfection (see FIGS. 21 and 22). However, in some embodiments, only the 2-1-1 waveform may outperform Expifectamine in the dual plasmid groups (see FIG. 23). All treatment groups across all plasmid experiments may be significantly greater in transfection efficiencies than the MEH protocol (BTX 8×20 ms) control.

Transfection efficiency may be lower in the GFPx+RFPx groups compared to single plasmids for all treatments other than Expifectamine (see FIG. 23). The majority of transfected cells may express both proteins with smaller subpopulations expressing either GFPx or RFPx. Additionally, when only one plasmid was transfected, it may be more than twice as likely to express RFPx than GFPx (2.28% vs 1.04%) when averaged across all bipolar pulse groups. This pattern may be reversed in the Expifectamine group (0.22% vs 2.50%) while the MEH (0.03% vs 0.04%) and untreated controls (0.00% vs 0.00%) may have approximately equivalent expression levels. When only a single plasmid is administered, GFPx may yield a higher transfection efficiency than RFPx. Each of these single plasmid groups may outperform co-transfection.

Embodiments of the present disclosure provide bipolar waveforms, which may be used, for example, to facilitate transfection in a clinical setting where induction of a necrotic wound would be counterproductive. In some embodiments, a field strength of, for example, 1500 V/cm may be used, as it may be the highest field strength that maintains an average viability of greater than 85% with a 2-1-2 continuous waveform. However, the present disclosure is not limited thereto. For example, this waveform may be used clinically for the treatment of equine melanoma (e.g., 2500V/cm, 0.02 s dose) without the need for general anesthesia or neuromuscular paralytics. For example, charge balanced asymmetric waveforms may induce less muscle stimulation compared to asymmetric non-charge balanced waveforms.

In some embodiments, a continuous waveform may yield lower viability (see FIGS. 16, 19, and 21 to 23) while burst balanced, cycle balanced, and symmetric burst waveforms may yield sequentially increasing viability. The MEH treatments may be among the highest performing treatment groups in terms of viability, but this protocol may result in the lowest transfection efficiency (0.53%). The bipolar waveform (1-1-0.05 continuous) with the lowest transfection efficiency (2.58%) may result in a 4.9× increase in transfection efficiency over the MEH protocol (see FIGS. 19 and 20). The burst balanced waveform (2-1-1 burst balanced) may yield a 9393× increase in transfection efficiency over the MEH protocol using GWiz GFP. This may be achieved with only a 5.6% reduction in viability compared to the MEH protocol. This indicates that field strengths (and applied voltages) may be significantly reduced while achieving similar in vivo transfection to established MEH protocols.

Qualitatively, imaging of cells 24 hours post transfection with GWiz GFP and RFPx plasmids (see FIG. 16) may align with the expected outcome based on the single plasmid transfection results in which protein expression is relatively low in the MEH protocol but sequentially enhanced in 2-1-2 continuous, 2-1-2 symmetrical burst, 2-1-1 cycle balanced, and 2-1-1 burst balanced groups (e.g., see FIGS. 15C to 15F). In all groups, the morphology of the cells may appear normal without blebbling or other signs of stress indicating induction of apoptosis. Quantitatively (see FIGS. 21 to 23), the most efficient bipolar pulse waveforms may significantly outperform the MEH protocol in single plasmid transfection. This may yield a 27,654× increase in transfection efficiency for GFPx (see FIG. 21) and a 16,692× increase in transfection efficiency for RFPx (see FIG. 22).

Transfection efficiencies may be substantially lower in groups receiving GFPx and RFPx simultaneously, despite all groups receiving the same quantity of DNA. This may result in a reduction of approximately 40% between the single plasmid and dual plasmid groups. It has been previously shown that the addition of a small empty plasmid may significantly enhance transfection efficiencies of larger plasmids through a process in which the smaller plasmids facilitate transmembrane transport of the larger molecule, but it has also been shown that transfection rates may decrease as plasmid size increases. In some embodiments, plasmids may be generated from the same backbone and contained approximately the same number of base pairs, so two similarly sized, larger plasmids may be more difficult for the membrane to diffuse as compared to one larger and smaller plasmid. For example, the plasmid shape and concentration as well as the time between plasmid treatment may alter the transfection efficiencies of different plasmids at different rates.

Bipolar waveforms may be substantially better at transfecting multiple plasmids than the MEH protocol. The bipolar waveform with the cotransfection efficiency (2-1-2 continuous) may yield a 6161× increase over the MEH protocol for GFPx+RFPx (see FIG. 23) and the highest cotransfection efficiency (e.g., 2-1-1 Burst Balance) may yield a 17,132× increase in cotransfection efficiency over the MEH protocol.

Lipidic delivery via Expifectamine may also outperform the MEH protocol on transfection efficiency (see FIGS. 21 to 23) and may achieve similar results to the bipolar pulse waveforms in dual plasmid cotransfection efficiency (e.g., 22.89% for Expifectamine and 28.56% for 2-1-1 burst balance). While there may be some advantages for using lipidic delivery in in vitro applications (e.g., ease, consistency, etc.), there may also be challenges in using lipid delivery in vivo due to poor health/transfection outcomes resulting from immunological responses, off target effects, and systemic toxicity. Immunotoxicity has been widely observed when utilizing lipoplexes (nonviral lipidic DNA carriers) in vivo as it may stimulate the production of inflammatory cytokines in many systems. Lipid delivery systems may also amplify severity of off target delivery as the lipids and their carries, being higher molecular weight, may accumulate in off target tissue when used in vivo. Lipoplexes may also be toxic to cells (cytotoxic) due to imbalanced charge ratios between the catatonic lipids and nucleic acids present in vivo. Additionally, each cell type may maintain a varying degree of sensitivity toward lipids, making lipidic delivery less universally applicable in vivo.

In some embodiments, the three highest transfection efficiencies may be found with asymmetrical waveforms. There may be several potential explanations for this phenomenon. For example, there may be a cumulative electrokinetic effect on negatively charged DNA molecules which leads to an increase in probability that DNA molecules will contact the cell membrane or that DNA is driven across concentration gradients. Burst balanced waveforms may be more efficient at transfection than cycle balanced waveforms, despite both waveforms having a neutral charge balance. Cycle balanced waveforms may result in relatively small movement (translation) of the DNA molecules due to the rapid recovery of charge balance within the waveform. In contrast, the burst balanced waveforms may maintain some degree of charge imbalance for the first half of the waveform. This may lead to larger translational movement of DNA molecules. Similarly, there may be differences in the net change in transmembrane potential during delivery of these different waveforms leading to more efficient generation of electro-pores for the burst balanced waveforms.

The two bipolar waveforms with the lowest transfection efficiencies may be asymmetric (e.g., 2-1-1 continuous and 1-1-0.05 continuous). This may indicate that timing is an important factor, as the continuous waveforms spread pulses out evenly over the entire treatment (e.g., 25 seconds) while the burst balanced and cycle balanced waveforms may be delivered in rapid successive pulses separated by, for example, 0.5 seconds. This may indicate that the dominant phenomena leading to enhanced transfection outcomes are occurring on microsecond timescales.

There may be some evidence to suggest that changes in pore resealing kinetics may be a factor when comparing MEH protocols and biopolar nanosecond waveforms. For traditional monopolar pulses (e.g., 100 μs or longer), pore resealing may take several minutes with shorter pulses (e.g., 100 μs), resulting in faster membrane resealing than longer ones (e.g., 2 ms). Both may reseal on the order of minutes. Bipolar nanosecond pulses may also exhibit similar resealing times as short pulses and longer pulses. This may suggest that there would be little to no difference in the opportunity for DNA uptake.

Bipolar nanosecond waveforms may exhibit no meaningful difference in resealing times, and bipolar nanosecond pulses may also reduce uptake of Ca²⁺ compared to monopolar nanosecond pulses by reducing number of pores formed (e.g., “bipolar cancellation effect”). These results may suggest that pore resealing in bipolar nanosecond pulses is similar to or faster than resealing for monopolar nanosecond pulses. It is thus unlikely that pore resealing is the catalyst for enhanced transfection efficiency in bipolar nanosecond pulses.

As another example, rather than resealing slower, bipolar pulse waveforms may create more/larger pores due to their rapid delivery. Energy may be delivered in smaller time periods resulting in less energy discharge between bursts. This may allow for a higher peak transmembrane potential to be reached and greater pore density.

Example embodiments of the present disclosure provide bipolar waveforms, which may be used, for example, in clinical transfection applications targeted at reducing negative effects of traditional electroporation protocols such as pain and intense muscle contractions. In some embodiments, burst balanced waveforms may have a high transfection efficiency, which results in a 9393× and 17132× increases in single plasmid and dual plasmid transfection versus traditional MEH protocols. Accordingly, bipolar waveforms may be a promising approach for in vivo gene transfection without the associated negative side effects.

Example embodiments of the present disclosure may provide bipolar microsecond electrical pulses. For example, bipolar microsecond pulses may offer substantial promise for DNA delivery via reversible electroporation. Several gene-related therapeutics such as DNA vaccines may be hindered by poor cellular uptake. Example embodiments of the present disclosure aim to overcome this by improving DNA delivery.

Bipolar microsecond and sub-microsecond pulsed electric fields may have several advantages over longer duration monopolar pulses such as, for example, significant reductions in muscle stimulation and perceived pain, which may enable their use in clinical applications. Some embodiments of the present disclosure may optimize treatment parameters to enhance DNA uptake in a 3D tissue model. For example, 3D tissue models may be subjected to microsecond pulsed electric field treatments with various waveforms, doses, and delivery rates. Small molecule uptake and viability may be evaluated in search of optimal outcomes. Computational models may then be used to derive reversible and lethal thresholds for each treatment group. DNA transfection may then be evaluated for a subset of optimal parameters and compared to traditional electroporation protocols.

In some embodiments, a 2-1-2 waveform with a 1 ms dose delivered at a rate of 100 μs/s may result in a highest number of transfected cells, yielding a 7730% increase over traditional monopolar pulse protocols.

The SARS-CoV-2 pandemic has highlighted the overwhelming importance of advanced non-viral nucleic acid-based vaccines, most prominently, mRNA vaccines. These vaccines may offer rapid development and production capabilities, and since they do not contain components of infectious viral agents, may have an extremely low risk of infection or insertional mutagenesis. However, it may be difficult to target specific tissues with mRNA vaccines, and the molecule may degrade quickly once in the body, necessitating encapsulation in a lipid nanoparticle and a need for repeated administration to maintain effective protein levels. In addition, mRNA formulations may require a cold storage chain creating logistical issues. DNA vaccines may offer the same primary advantages of rapid development and production, but may also be significantly more stable, leading to more protein generation per molecule and the ability to be stored at room temperature. Despite these advantages over mRNA, DNA vaccines have yet to become a clinical standard due to poor cellular uptake and therefore unpredictable expression in vivo.

As discussed above, electroporation is a process in which pulsed electric fields (PEFs) may be utilized to induce nanoscale pores throughout the cell membrane. Electroporation may only occur when the local electric field surpasses a critical threshold, resulting in temporary defects which permeabilize the membrane to macromolecules that are otherwise incapable of entering the cell. This process may be termed reversible electroporation (RE) due to the transient nature of cell membrane permeability and relatively high levels of cell survival and DNA expression following treatment. However, if cells are exposed to an electric field intensity which surpasses a higher, lethal threshold, a rapid decline in cellular viability may be induced by a process known as irreversible electroporation (IRE). These reversible and irreversible thresholds may be significantly impacted by, for example, pulse width, dose, and temperature.

Traditionally, reversible electroporation has been implemented for drug and gene delivery applications by employing monopolar pulses on the order of, for example, 100 μs-10 ms. This has enabled electroporation to be used as a delivery vehicle for DNA, which may increase transfection by approximately 500× in vitro versus non-electroporated controls. However, these pulse durations may induce intense muscle stimulation and pain in vivo, potentially limiting their clinical utility in an outpatient setting.

Bipolar pulses may be used for pre-clinical irreversible electroporation ablations without inducing muscle stimulation. These pulses may also maintain a homogeneous field distribution despite the surrounding heterogeneity of the tissue environment, enabling more predictable treatment outcomes. Bipolar pulses may also offer a new set of parameters including, for example, waveform symmetry, pulse duration, pulse frequency, and dose, which can be adjusted to affect cellular responses to pulsed electric fields where both the reversible and irreversible thresholds fall within the treatment zone. Ultrashort (e.g., 0.5-2.0 μs) bipolar pulses may have the potential to resolve many issues related with traditional long duration (e.g., 100 μs-10 ms) pulses such as, for example, pain, muscle stimulation, and cardiac complications. Example embodiments of the present disclosure may provide methods, systems, and devices for optimizing bipolar pulses and their associated parameters to enable clinical use of reversible electroporation for DNA vaccine delivery.

To develop effective in vivo transfection protocols for DNA vaccine delivery, it may be helpful to identify reversible and irreversible thresholds for a given pulse sequence as well as the development of applicators that expose tissues to fields that surpass the reversible threshold while limiting those below the irreversible threshold. For example, pulses with durations less than or equal to 2 μs may be optimized for electroporation-mediated delivery of a model small molecule, propidium iodide (PI), through a large parameter sweep of different waveform parameters (e.g., pulse duration, asymmetry, energy delivery rate, dose, etc.) Dose may play an important role in affecting the difference between lethal and reversible thresholds. The ability to induce stable gene transfection may be evaluated using a subset of these protocols, which may be compared to traditional electroporation protocols. For example, electroporation protocols according to embodiments of the present disclosure may result in a 7730% in the number of transfected cells versus traditional electroporation controls, which indicates that ultrashort pulse duration bipolar waveforms may be a promising avenue for in vivo DNA vaccination.

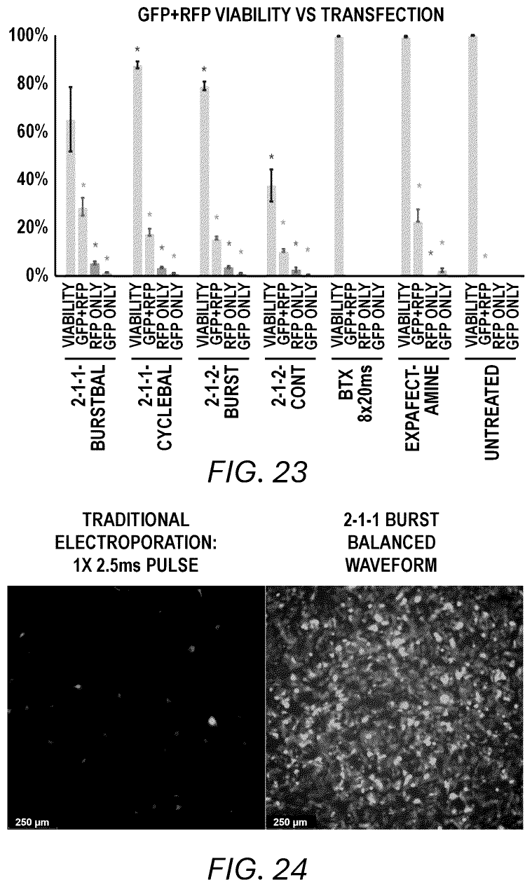

FIG. 24 is a schematic diagram illustrating transfection efficiency for electroporation processes, according to some embodiments of the present disclosure. In particular, FIG. 24 illustrates the transfection efficiency for a traditional electroporation process (on the left side) versus the transfection for an electroporation process according to some embodiments of the present disclosure (on the right side).

Referring to FIG. 24, VERO cultures may be transfected with EGFP (enhanced green fluorescent protein) RNA via a traditional electroporation process (e.g., 1×2.5 ms pulse) and an electroporation process according to some embodiments of the present disclosure (e.g., 2-1-1 burst balanced waveform). As shown in FIG. 24, transfection efficiency may be significantly greater for the 2-1-1 burst balanced waveform than the 1×2.5 ms pulse.

For applications where cells are used for biomanufacturing, it may be helpful to modify the cells by delivering nucleic acids (RNA, DNA, etc.) or other pharmaceutical agents which do not easily cross the cell membrane. To generate high yields, it may be helpful for the cells to remain completely viable and metabolically active for at least 10-24 hours following transfection and typically it is desirable to maintain long term viability. A challenge in the use of electroporation for transfection may be that this process is typically highly lethal with greater than 50% of cells sometimes destroyed during the transfection process. Mammalian cells (HEK293) may be transfected with two DNA plasmids using protocols with two different waveforms (2-1-2 Continuous and 2-1-1 Burst Balance). In this case, transfection efficiencies may be 6,161 to 17,132 times greater than the efficiency of traditional electroporation protocols. The unique waveforms and use of temperature control may significantly improve cell viability compared to traditional approaches. A challenge, however, may be that this process relies on the use of cuvettes to process 100-1000 μL of samples at a time (0.1-1.0 million cells per batch). This may be a highly manual process, which limits the number of cells that can be processed and the quantity of manufactured reagents which can be achieved.

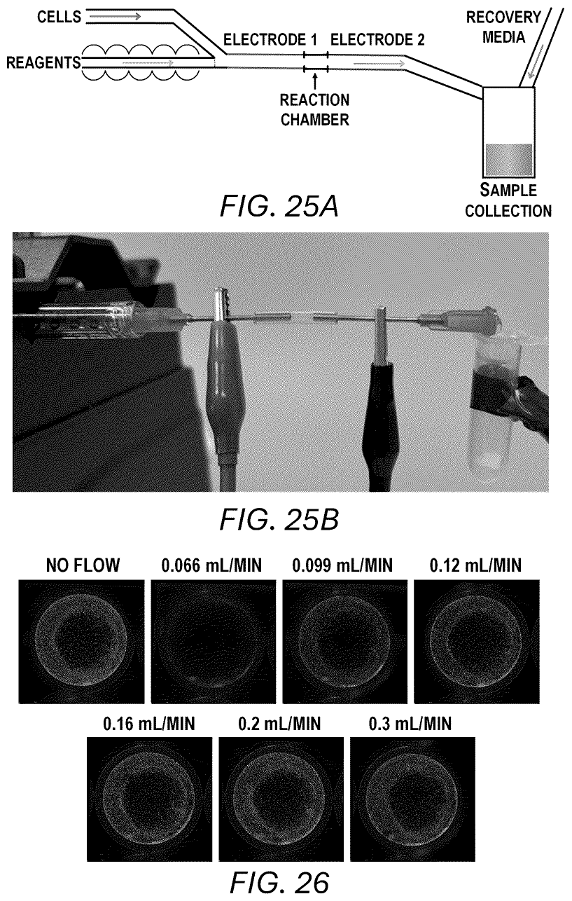

FIG. 25A is a schematic diagram illustrating a flow through device for biomanufacturing, according to some embodiments of the present disclosure. FIG. 25B is a schematic diagram illustrating a demonstration of a flow through process, according to some embodiments of the present disclosure.

Referring to FIGS. 25A and 25B, cells and reagents may be mixed together prior to entering a reaction chamber including two electrodes separated by an insulating region. After leaving the reaction chamber, cells may be collected in a vessel.

In some embodiments, electrical energy may be continuously administered, which may be highly amenable to a flow through process in which cells and reagents are continuously flown through a reaction chamber. Cells may be exposed to the electric field only for the duration that they are within the reaction chamber. In this way, the flow rate and energy delivery rate may be used to control the electrical dose to which cells are exposed. The geometry of the reaction chamber may be used to adjust the volume of cells which are processed in a given timeframe. Similarly, multiple reaction chambers may be used in parallel to multiply production yields.

An advantage of the above approach may be that cells and reagents can be held separately until immediately before transformation. This may enable the cells to be maintained at physiological temperatures (or other ideal temperatures) while reagents which may be temperature sensitive are held at lower temperatures (or other ideal temperatures). The reaction chamber may similarly be heated or cooled to achieve optimal conditions for delivering genetic or large molecule payloads.

Cells may be passaged via normal protocols and suspended in EMEM (10% FBS, 1% PenStrep). Cells may be counted and suspended in a 2e6 cells/mL, then 20 μg/million cells of GFP plasmid may be added to the cell suspension. Cell+reagent mixture may then be pumped through the reaction chamber where cells are exposed to various electric field intensities, pulse waveforms, and electrical doses. Following processing, 500 microliteres of the cell suspension may be added to each well in a 12 well plate in addition to an equal volume of recovery media containing modified EMEM (25% FBS, 1% PenStrep). The cells may then be incubated for 24 hours prior to further analysis.

COMSOL Multiphysics may be used to narrow in on parameters that wouldn't thermally kill cells. Parameters may be optimized for 1500 V/cm across all gap distances and needle sizes. Most of the heating may happen at the edges of the needle and then the fluid may cool down as it passes through the chamber. Needle gauges may range from 14-27 AWG. In some embodiments, the smaller the gap and the smaller the gauge, the less heating there may be of the cells. The electric field may be uniform across the gap. Example treatment parameters are shown in Table 5.

TABLE 5

| Needle Gauge | T1 (°C) | T2 (°C) | T3 (°C) | Tavg (°C) |
|---|---|---|---|---|
| 14 | 79 | 80 | 79 | 79 |
| 15 | 69 | 75 | 73 | 72 |
| 18 | 40 | 39 | 39 | 39 |
| 20 | 45 | 42 | 42 | 43 |

Temperature may be measured and heating less than 45° C. may be selected. Treatments may include, for example, 750 V pulses, a 1-1-1 waveform and a 0.02 s dose administered at 1000 μs/s with a flow rate of 0.074 mL/min through a 5 mm gap.

In some embodiments, the best performance may be achieved with 18 and 20 AWG tubing and electrodes.

Conductivity of the media may be evaluated with a 5 mm gap and a flow rate of 0.033 mL/min, as shown in Table 6.

TABLE 6

| Conductivity | | | | |
|---|---|---|---|---|
| (S/m) | T1 (°C) | T2 (°C) | T3 (°C) | Tavg (°C) |
| 0.01 | 17 | 16 | 16 | 16.3 |
| 0.023 | 17 | 16 | 16 | 16.3 |
| 0.185 | 18 | 19 | 19 | 18.6 |
| 0.414 | 21 | 22 | 21 | 21.3 |
| 0.785 | 28 | 27 | 27 | 27.3 |
| 1.564 | 40 | 40 | 40 | 40 |

All conductivities may result in temperatures less than 45° C., indicating that the device is capable of working with a wide range of buffers.

Baseline experiments with cells in media may then be conducted to evaluate if the act of flowing cells through the device had an effect on viability. It was found that flowing cells through the device may have a minimal impact on viability and that higher flow rates may result in greater numbers of cells collected.

FIG. 26 is a schematic diagram illustrating the viability of cells processed through the flow through device without an electric field, according to some embodiments of the present disclosure.

Referring to FIG. 26, viability may be preserved as cells are processed through the device without an electric field. Live/dead (green/red) assay following flow through at flow rates between 0.006 and 0.3 mL/min may indicate that cells retained a high viability and larger volumes of cells were collected as the flow rate was increased.

Experiments may be conducted to determine the effect of flow rate and waveform on transfection efficiency and cell viability. Initial experiments demonstrated that transfection efficiencies of 3 to 15.5% could be achieved with flow rates of 0.066 to 0.3 mL/min. Optimization of pulse waveforms with these baseline conditions may result in an increase of 16 to 53% transfection efficiency. The highest efficiency may be achieved with a 2-1-1 burst balanced waveform. Higher flow rates may result in a decrease in efficiency likely due to cells being exposed to a lower dose. This may be overcome by increasing the rate of energy delivery from 1000 μs/s to a higher rate to increase the overall dose cells receive as they transit the reaction chamber.

Accordingly, the flow through transfection device was validated. This device may include a 5 mm reaction chamber through which 750 V waveforms may be administered as cells continuously transited the reaction chamber. Transfection efficiencies up to 53% may be observed, and the highest efficiency may be achieved with a 2-1-1 burst balanced waveform. Numerical simulations and experimental data show that device parameters may be optimized to maintain joule heating effects at or below 45° C. and that active temperature control could be used to maintain optimal temperatures for transfection to occur.

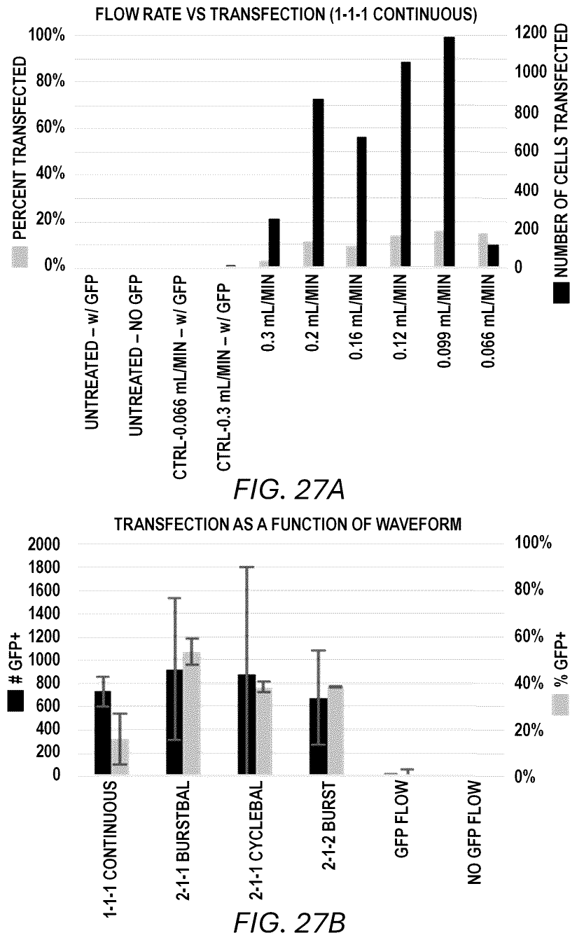

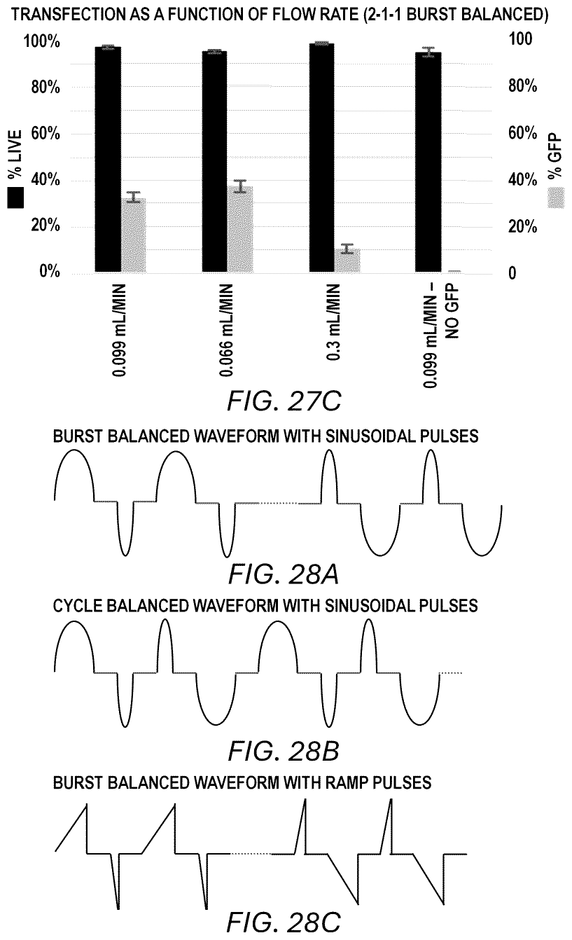

FIGS. 27A to 27C are example graphs illustrating transfection in a flow through device and optimization of flow rates and pulse waveforms, according to some embodiments of the present disclosure.

Referring to FIGS. 27A to 27C, initial experiments may demonstrate that transfection efficiencies of 3 to 15% can be achieved (see FIG. 27A). Optimization of the pulse waveform may increase this efficiency to 16-53% with 750 V, 1000 us/s, 1.67 ms dose, 0.099 mL/min (see FIG. 27B). Higher flow rates may result in a decrease in efficiency (see FIG. 27C).

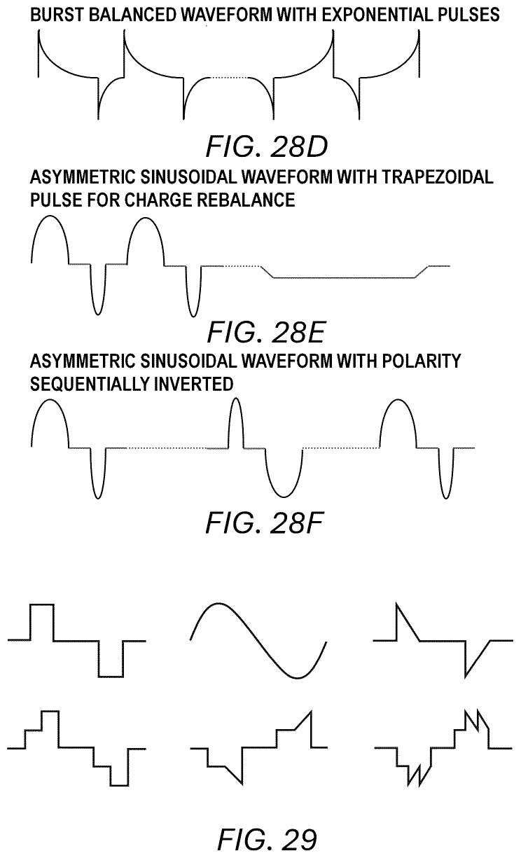

FIGS. 28A to 28F are schematic diagrams illustrating example waveforms including electrical pulses, according to some embodiments of the present disclosure. The waveforms shown in FIGS. 28A to 28F may also be referred to as asymmetric charge-balanced electrical waveforms.

Referring to FIGS. 28A to 28F, example charge balanced waveforms may include a burst balanced waveform with sinusoidal pulses (see FIG. 28A), a cycle balanced waveform with sinusoidal pulses (see FIG. 28B), a burst balanced waveform with ramp pulses (see FIG. 28C), a burst balanced waveform with exponential pulses (see FIG. 28D), an asymmetric sinusoidal waveform with a trapezoidal pulse to achieve overall charge balance (see FIG. 28E), and an asymmetric sinusoidal waveform where polarity is sequentially inverted to achieve charge balance (see FIG. 28F).

Referring to FIGS. 15A to 15F and 28A to 28F, charge balanced waveforms according to embodiments of the present disclosure may be constructed from a number of different pulse geometries. For example, the pulses may be square pulses (see FIGS. 15B to 15F), sinusoidal pulses (see FIGS. 28A and 28B), ramp pulses (see FIG. 28C), exponential decay pulses (see FIG. 28D), or shapes made from combinations of these geometries with different amplitudes. As used herein, the amplitude of a pulse (or a phase thereof, such as a positive or negative phase) may also be referred to as the magnitude of the pulse, where the magnitude indicates the absolute value of the amplitude of the pulse (or the phase thereof). In some embodiments, the waveforms may be burst balanced where an asymmetric waveform is repeated for half of the burst and inverted for the second half of the burst (see FIGS. 15F and 28A). In some embodiments, the waveforms may be cycle balanced where the waveform is sequentially inverted to continuously achieve charge balance (see FIGS. 15E and 28B). In some embodiments, a series of asymmetric waveforms may be administered, and charge balance may be restored by intermittent delivery of different amplitude, opposite polarity waveforms of a different shape (see FIG. 28E). For example, a series of asymmetric sinusoidal waveforms may be administered with a net positive charge, and then a single longer duration trapezoidal pulse may be administered to regain charge balance prior to administration of more asymmetric sinusoidal waveforms (see FIG. 28E). In some embodiments, each sequential asymmetric waveform administered may have alternating polarities (see FIG. 28F) to maintain charge balance when individual monopolar waveforms or bipolar waveforms are administered.

Delivery of the pulse waveform may include selecting a number of cycles in each burst, a number of bursts delivered, selecting the charge-balanced cycles or charge-unbalanced cycles, and, in the case of non-charge-balanced cycles, selecting charge-balanced bursts or non-charge-balanced bursts. A delivered waveform with charge-balanced cycles may have positive and negative pulses of the same voltage magnitudes. The positive and negative pulses may have the same duration of, for example, 0.1 to 10 microseconds, or may have different durations, each with a duration of, for example, 0.1 to 10 microseconds. In some embodiments, a total on-time duration of positive pulses may be the same as a total on-time duration of negative pulses.

Delivering to the target tissue a series of electrical pulses may include, for example, delivering a burst energized for 0.2-1000 microseconds, 1 to 100 times, with a 0.1 to 100 Hz repetition rate between bursts, whereby a zone of reversibly electroporated cells is produced.

Delivering to the target tissue the series of electrical pulses may include, for example, delivering pulses for 0.2 to 2000 microseconds by sequentially repeating the series of alternating positive and negative pulses. Delivering to the target tissue the series of electrical pulses may include delivering pulses at a voltage between 1 and 10,000 V. Delivering to the target tissue the series of electrical pulses may include delivering pulses at a frequency of 0.01 to 1,000,000 Hz (e.g., between 0.1 and 1000 Hz). Delivering to the target tissue the series of electrical pulses may include delivering pulses sequentially 2 to 10,000 times, (e.g., 2-1000 times).

FIG. 29 is a schematic diagram illustrating example waveforms including electrical pulses, according to some embodiments of the present disclosure.

Referring to FIG. 29, charge balanced waveforms according to embodiments of the present disclosure may be constructed from a number of different pulse geometries, some examples of which are shown.

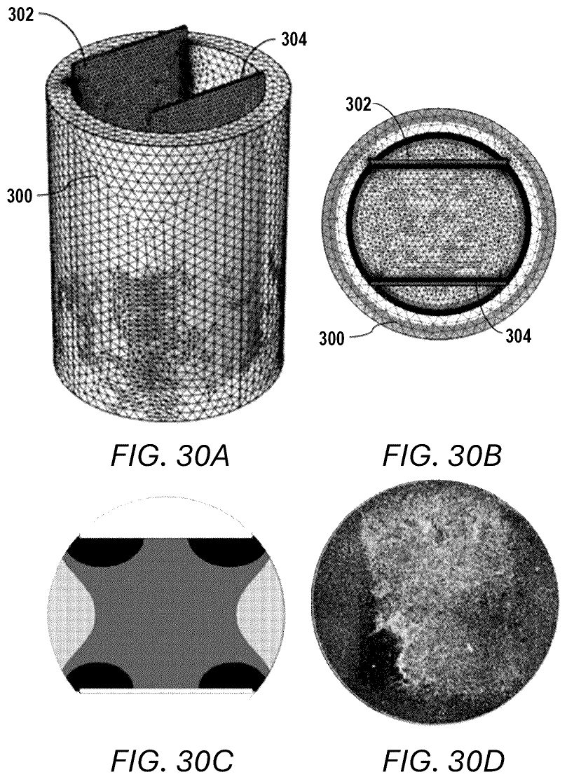

FIGS. 30A to 30D are schematic diagrams illustrating stages of an in vitro technique for treating cells in a 3D tissue model, according to some embodiments of the present disclosure. FIG. 30A is an isometric view of a cell culture well 300 with concentric ring and pin electrodes 302, 304 inserted. FIG. 30B is a top-down view of a cell culture well with ring and pin electrodes inserted. The electrodes may extend from outside of the well to the bottom well surface and enable the delivery of electrical energy to the cells within the tissue model. FIG. 30C illustrates a finite element simulation showing the electric field distribution within the well when 750V is applied between ring and pin. Isocontour boundaries between shaded regions depict electric field strengths of 2000 V/cm, 1750 V/cm, 1500 V/cm, and 1250 V/cm. FIG. 30D is an image showing adherent cells which were exposed to a sufficient electric field to become electroporated adjacent to unaffected cells. The cells in FIG. 30D were exposed to 750V pulses, which indicates that reversible electroporation may occur at 1500 V/cm and irreversible electroporation may occur at 1750 V/cm.

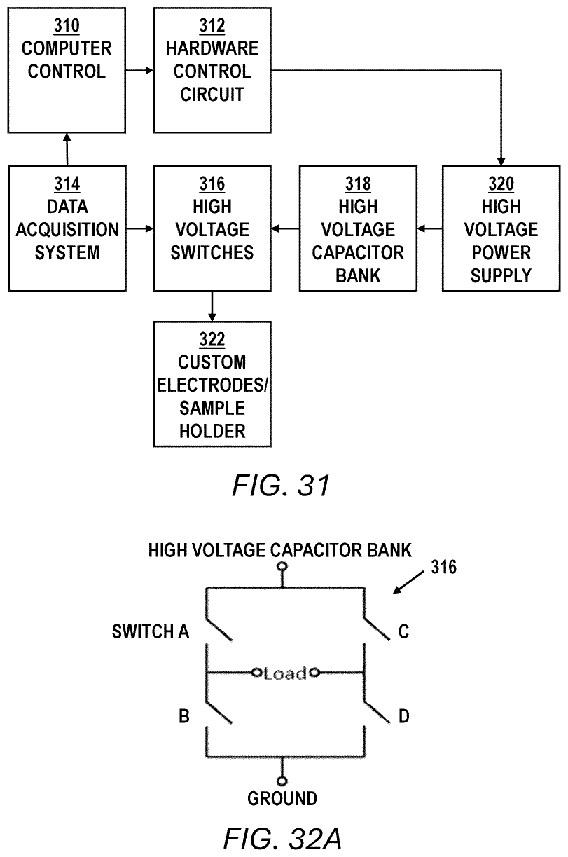

FIG. 31 is a schematic block diagram illustrating a system for delivering electroporation treatment using pulse waveforms, according to some embodiments of the present disclosure.

Referring to FIG. 31, a pulse delivery system may include a computer controller 310 coupled to a hardware control circuit 312 and a data acquisition system 314. The computer controller 310 may include a graphical user interface (GUI). The hardware control circuit 312 may include a microcontroller or field programmable gate array (FPGA), which is connected to pulse delivery hardware (e.g., high voltage switches) 316 and a high voltage power supply 320. In some embodiments, hardware control circuit 312 may also be connected to additional circuitry such as a heartbeat synchronization, arc detection, over current detection, safety interconnects, logical controls, sensors (e.g., temperature sensors) and/or an emergency on/off switch. High voltage switches 316 may deliver pulse waveforms to custom electrodes 322 in contact with or adjacent targets cells or tissue. A high voltage capacitor bank 318 may be connected between the high voltage power supply 320 and the high voltage switches 316.

After parameters are selected (e.g., using the graphical user interface), computer controller 310 may communicate the appropriate pulse waveform parameters (e.g., voltage, pulse lengths, number of pulses, number of bursts, repetition rate, waveforms, etc.) to the hardware control circuit 312 (e.g., microcontroller/FPGA). The hardware control circuit 312 may wait for an appropriate command (e.g., foot petal or button press) to start the selected treatment.

Once treatment is initiated, the hardware control circuit 312 may turn on the high voltage power supply 320, which charges the high voltage capacitor bank 318 to an appropriate level. High voltage switches 316 may then deliver pulse waveforms to custom electrodes 322 in contact with or adjacent target cells or tissue. The system may deliver the treatment and monitor for emergency stop conditions (e.g., arc, over current, button press). It may also monitor the treatment for voltage and current and adjust the high voltage power supply 320 to adequately maintain the appropriate conditions.

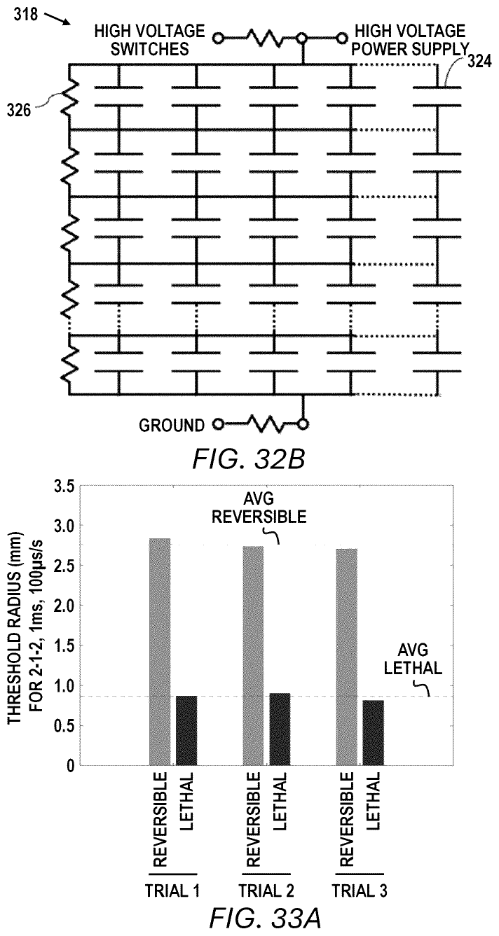

FIG. 32A is a schematic diagram of a circuit for generating symmetric and asymmetric pulse waveforms, according to some embodiments of the present disclosure. FIG. 32B is a schematic diagram of a capacitor bank architecture, according to some embodiments of the present disclosure.

Referring to FIGS. 32A and 32B, high voltage switches 316 may have an H-Bridge configuration which includes a minimum of four MOSFET or IGBT switching stages (switches A, B, C, D). Each switching stage may in turn be a series stack of 1-100 lower voltage MOSFETs or IGBTs designed to open and close at the exact same time. The four switching stages may be configured in a bridged configuration such that Switch A and Switch D close simultaneously to generate a positive polarity pulse and Switch C and Switch B close simultaneously to generate a negative polarity pulse.

These electroporation methods may deliver high voltages and high currents. Instantaneous power can be in excess of, for example, 0.4 megawatts. A typical power supply may not deliver both high voltage and high current. To accomplish this, a low-current, high voltage power supply 320 (see FIG. 31) may be used to charge a capacitor bank 318. Capacitors which can withstand 2-10 kV and store large quantities of charge may be expensive, and sometimes not available in the capacitance values needed (e.g., 15-200 microFarads). To achieve the desired output, a capacitor bank 318 may be used that includes a stack of series connected capacitors which are rated, for example, for 1000-1500 V each. By connecting them in series strings, the voltage which the capacitor bank 318 can withstand may be increased. To increase the capacitance value of the bank 318, multiple series strings may be connected in parallel. To protect any individual capacitor 324 from experiencing a voltage above its rated voltage, a high value resistor 326 (e.g., 0.5-100 megaOhms) may be connected in parallel with each capacitor. These resistors may also help discharge the capacitor bank 318 to safe levels once the power has been turned off.

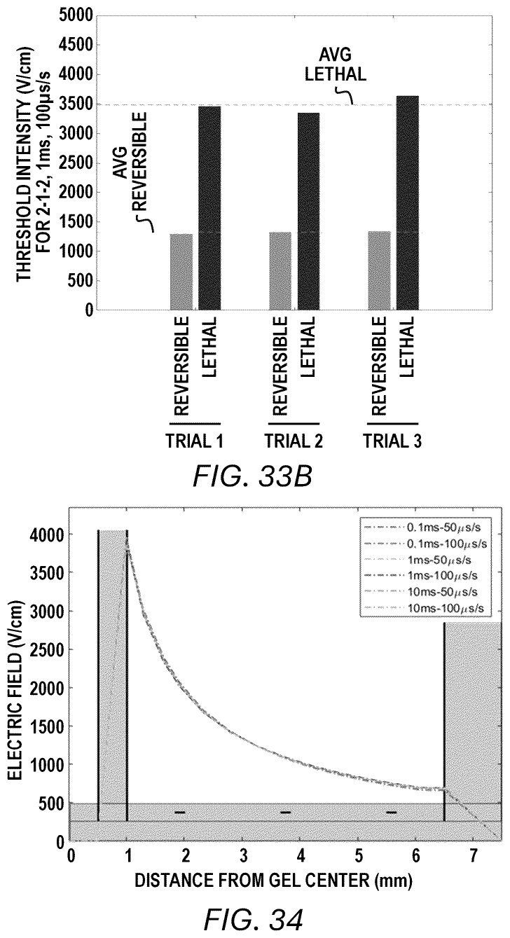

FIGS. 33A and 33B are example graphs illustrating results of treatment parameters, according to some embodiments of the present disclosure. For example, the 2-1-2 waveform, 1 ms dose, 100 μs/s treatment may be optimal for the parameters tested. This may result in reversible and lethal radii of 2.762±0.170 mm and 0.867±0.116 mm, respectively. These may correspond to field intensities of 1322±62.67V/cm and 3486±358.9V/cm, respectively.

FIG. 34 is an example graph illustrating an electric field in a ring-and-pin electrode, according to some embodiments of the present disclosure.

Electrodes (e.g., see the custom electrodes 322 in FIG. 31) used to deliver pulse waveforms according to embodiments of the present disclosure may have various configurations. For example, two independent electrodes (source and sink) may be inserted into the target cells or tissue. Three or more independent electrodes may be inserted into the target cells or tissue and combinations of one or more electrodes may act as an electrical source and a combination of one or more electrodes may act as electrical sinks. One electrode may act as the electrical source and is inserted into the target cells or tissue and a grounding pad placed on the skin may act as the electrical sink. Two or more electrodes may be inserted into the target cells or tissue and energized simultaneously (all electrical sources) and a grounding pad on the skin may act as the electrical sink. Two or more electrodes may be inserted into the target cells or tissue and energized independently and a grounding pad on the skin may act as the electrical sink. One electrode may be an array of smaller conductors acting as an electrical source and a grounding pad on the skin may act as the electrical sink. One electrode may be an array of smaller conductors acting as an electrical source and a separate electrode on the same probe may act as an electrical sink. Two or more electrode arrays of smaller conductors may act as electrical sources and sinks.

The electrodes may be used to deliver the compound (e.g., target molecule(s)) directly to the target site. For example, the compound may be administered to an environment surrounding the target cells or tissue. The compound may be delivered systemically, or the compound may exist naturally in the target tissue or media. The compound may be a fluid cooled at or below body temperature, or may be warmed at or above body temperature. The compound may be a highly conductive fluid (0.3 to 2.0 S/m) such as phosphate buffered saline, normal saline, Lactated Ringers solution, Viaspan (UW solution), or another conductive isotonic solution. The compound may have a low conductivity (0.001 to 0.3 S/m) such as deionized water or deionized water with dissolved solids, proteins, or ions required to make it isotonic. The compound may contain DNA, RNA, RNAi, SIRNA, genes, endonucleases, amino acids, polypeptides, proteins, or other biological target molecule(s) which change the behavior or genetic code of cells. Example proteins include Cas3, Cas8a, Cas5, Cas8b, Cas8c, Cas10d, Cse1, Cse2, Csy1, Csy2, Csy3 GSU0054, Cas10, Csm2, Cmr5, Cas10, Csx11, Csx10, Csf1, Cas9, Csn2, Cas4 Cpf1, C2c1, C2c3, C2c2. CRISPR-Cas9, CRISPR-DR2, CRISPR-DRS, CRISPR-DR6, CRISPR-DR8, CRISPR-DR9, CRISPR-DR19, CRISPR-DR41, CRISPR-DR52, CRISPR-DR57, CRISPR-DR65, DRACO (Double-stranded RNA Activated Caspase Oligomerizer), zinc-finger nucleases, transcription activator-like nucleases. The target molecule(s) may include a chemotherapy intended to induce necrosis, apoptosis, or DNA damage. Example target molecule(s) include Anthracyclines, doxorubicin (Adriamycin) epirubicin (Ellence), Taxanes, paclitaxel (Taxol), docetaxel (Taxotere), 5-fluorouracil, Cyclophosphamide, Carboplatin, Docetaxel, Paclitaxel Platinum agents (cisplatin, carboplatin), Vinorelbine (Navelbine), Capecitabine (Xeloda), Liposomal doxorubicin (Doxil), Gemcitabine (Gemzar), Mitoxantrone (Novantrone), Ixabepilone (Ixempra), Albumin-bound paclitaxel (nab-paclitaxel or Abraxane), Eribulin (Halaven). The target molecule(s) may include molecules intended to give the target tissue a new biological function or induce the production of specific molecules. The target molecule(s) may include a drug intended to treat a specific disease or stimulate the production of a compound. Examples include up-regulation of the production of insulin by pancreatic cells in diabetic patients.

In some embodiments, waveforms may include a 2 μs positive, 1 μs delay, 2 μs negative phase structure which is repeated in a burst of 25 cycles to create an innately charge-balanced burst which delivers approximately 100 μs of energy. Waveforms may have cycles including a 2 μs positive, 1 μs delay, 1 μs negative phase structure which is repeated in a burst of 34 cycles to create an innately charge-unbalanced burst which delivers approximately 100 μs of energy. Waveforms may have cycles including a 2 μs positive, 1 μs delay, 1 μs negative phase structure which is repeated in a burst of 34 cycles where the duration of the positive and negative phases are switched with each successive cycle to create a charge-cycle-balanced burst which delivers approximately 100 μs of energy. Waveforms may have cycles including a 2 μs positive, 1 μs delay, 1 μs negative phase structure which is repeated in a burst of 34 cycles where the each cycle in the first half of the burst has a corresponding cycle in the second half of the burst where the duration of the positive and negative phases are switched to create a charge-burst-balanced burst which delivers approximately 100 μs of energy. Embodiments of the present disclosure are not limited to the above waveforms.

Pulse structures may be denoted by the compact notation (P-D-N) where P indicates the positive pulse length in microseconds, N indicates the negative pulse length in microseconds, and D indicates the delay in microseconds between successive pulses. As used herein, differences of 10% or more between P and N may constitute an unbalanced waveform, while differences less than 10% may constitute a balanced waveform. For example, (2-1-0.25) may represent an asymmetric pulse waveform with a 2 μs positive pulse, followed by a 1 μs delay, then a 0.25 μs negative pulse. As used herein, a pulse waveform including a positive pulse and a negative pulse separated by a delay (i.e., a pulse free delay) may also be referred to as an electrical pulse including first and second phases (i.e., positive and negative phases) separated by an interphase delay, where the first and second phases have opposite polarities. In other words, positive and negative pulses included in a pulse waveform (i.e., a biphasic pulse) may also be referred to as first and second phases of an electrical pulse. Further, as used herein, an asymmetric waveform that results in a net charge balance by an end of delivery may also be referred to as an asymmetric charge-balanced electrical waveform. The notation (2,−1,−2) may represent a symmetric waveform with alternating polarity 2 μs pulses separated by a 1 μs delay. In some embodiments, a 1 μs delay may be used between changes in pulse polarity.

The target tissues according to embodiments of the present disclosure may be mammalian cells, eggs, zygotes, embryos, Prokaryotes (bacteria or archaea), Eukaryotes (protozoa, algae, fungi), or yeast grown in in vitro culture or suspension. The target tissues may be cells in blood, serum, plasma, urine, bile, lymph, semen, or other biological fluids. The target tissues may be cancers growing in healthy organs. Example cancers include prostate cancer, breast cancer, liver cancer, kidney cancer, bladder cancer, bone cancer, brain cancer, skin cancer, and other cancers of the bone, blood, lymphatic system, or soft tissues. The target tissue may be diseased tissue or healthy tissue.

In some embodiments, an apparatus may be used for reversible electroporation. The apparatus may include therapeutic electrodes for insertion into target tissue cells, a computer controller for generating a pulse waveform, that is, a hardware controller for generating high voltage electrical pulses to the therapeutic electrodes in accordance with the generated pulse waveform, such that reversible electroporation can be controlled by selecting the charge-balanced cycles or charge-unbalanced cycles, and, in the case of non-charge-balanced cycles, selecting charge-balanced bursts or non-charge-balanced bursts.

The apparatus may also include a high voltage capacitor bank comprising four switches to create an H-Bridge configuration for delivering positive and negative polarity pulses from a single high voltage power supply. It may include two switches in a totem pole configuration for delivering positive and negative polarity pulses from a positive and negative power supply. The apparatus may include a transformer in conjunction with an H-Bridge or totem pole configuration to step up a lower voltage to a higher voltage.

The switches may include MOSFET, SiC MOSFET, IGBT, or relay switches. Each switch may include two or MOSFETs, SiC MOSFETs, IGBTs, or relays in series to increase the voltage that each switch can withstand. Each switch may include two or more MOSFETs, SiC MOSFETs, IGBTs, or relays in parallel to increase the current that each switch can deliver. Each switch may be made up of two or more components in parallel and two or more components in series to increase the voltage and current that each switch can deliver.

The apparatus may include a high voltage source connected to a capacitor bank of one or more capacitors in a series-parallel arrangement. The apparatus may include a high voltage source connected to a single high voltage capacitor.

In some embodiments, a method of electroporation may be used as part of a clinical workflow for gene transfection, electrochemotherapy, and/or other intracellular delivery of therapeutic substances. An appropriate anesthetic plane may be established to allow the physician to insert one or more therapeutic electrodes into the target cells or tissue. This tissue could be a tumor for oncology, healthy or poorly functioning pancreatic tissue for diabetes treatment, brain tissue for the treatment of Parkinson's or other neurological disorders, bone or bone marrow for immunotherapy, skin for the treatment of dermatitis, skin diseases, and infection or fertilized egg/zygote/embryo for genetic engineering purposes. The desired compound, e.g., gene sequence, silencing RNA, chemotherapy, CRISPR, and/or Cas protein, may then be injected into the patient either systemically through the vasculature or directly into the treatment zone. If injected directly into the treatment site, the therapeutic electrodes may be used as the delivery tool. The clinician may then insert electrodes into or around the target volume and delivers a series of electrical pulses that minimize cell death while improving uptake of large molecules into the cell.

Pulse bursts may include, for example, between 2 and 1000 electrical pulses which alternate in polarity between positive and negative voltage and current. Each pulse may be of equal duration and can be between 10 ns and 1 ms (e.g., between 100 ns and 10 μs). Each treatment may include the delivery of 1-1000 bursts (e.g., 1-100 bursts) delivered at a repetition rate between 0.001 Hz and 1000 Hz (e.g., between 0.1-10 Hz).

The treatment electrodes may be a pair (source and sink) of electrodes inserted directly into the treatment site, multiple pairs (sources and sinks), a single electrode and distant grounding pad, tweezers placed around the target tissue, or electrodes inserted into the vasculature designed to treat an entire organ or specific volume of tissue.

The clinician may use a software interface on the device to program the volume of tissue treated. The physician may select the type of treatment desired and the computer may automatically select the appropriate waveform to deliver. The physician may select which electrode configuration will be used and a graphical panel may display the anticipated treatment volume. As another example, the physician may increase or decrease the target volume and the computer may determine which voltages and pulse parameters should be used. The physician may then press a start button or foot pedal to initiate treatment using pulse waveforms according to embodiments of the present disclosure. The system may then automatically deliver the appropriate treatment and monitor the electrical properties (impedance, resistance, conductivity) of tissue. This information may be used to determine appropriate treatment endpoints as well as monitor for arcing, heating, or other deleterious effects. After the electrical bursts are delivered, the treatment may be finished and the physician may remove the electrodes and closes any incisions made.

In other embodiments, a method of electroporation may be used as part of a workflow for laboratory or in vitro gene transfection, electrochemotherapy, and intracellular delivery of other substances. Target cells may be treated in suspension, while they are adhered to the surface of a well plate, while they are in a 3D engineered tissue matrix (matrigel, collagen, agarose, etc.), or in situ in an ex-vivo organ.

Adherent cells may be released from the culture plate via trypsinization, centrifuged, washed, and suspended in a transfection buffer containing the desired molecule(s). The buffer/cell suspension may be injected into an electroporation cuvette containing source and sink electrodes on opposing sides of the cuvette. Electrodes may be separated by 0.1-10 cm (e.g., by 0.1-1 cm). A series of pulse bursts may then be delivered across the cuvette using pulse waveforms according to embodiments of the present disclosure. The cells may then be removed from the cuvette and returned to normal culture conditions.

The culture media above adherent cells may be removed and replaced with a transfection buffer containing the desired gene sequences or RNA. Electrodes may then be inserted into the culture well into contact with the bottom of the well plate. A series of electrical bursts are then delivered through the electrodes. The transfection buffer is then removed, and normal culture media is added to the cells. Alternatively, cells can be cultured in well plates which are engineered to have embedded electrodes along the sides/bottom of the well plate. This process is identical for cells grown in a 3D tissue model.

Embodiments of the present disclosure may provide methods, devices, and systems for using electrical pulses to permeabilize cells and tissues to increase uptake of molecular components to elicit cellular responses. Embodiments of the present disclosure may provide methods, devices, and systems for permeabilizing cells and tissues using bursts of high-frequency, bipolar electrical pulses having burst durations (i.e., burst widths) on the order of microseconds and duration of single polarities on the microsecond to nanosecond scale. In some embodiments, the electrical pulses may be delivered using (or via) an asymmetric charge-balanced electrical waveform. These pulses may be used to enhance membrane permeability or induce transient pores to form across the cell membrane, enhancing transport of compounds containing target molecule(s) across the cell membrane. For example, the target molecule(s) may be administered to an environment surrounding target cells or tissue, and the electrical pulses may be used to enhance membrane permeability or induce transient pores to form across the cell membrane(s) of the target cells or tissue, to thereby transiently enhance transport of the target molecule(s) across the cell membrane(s).

Unless otherwise defined, all terms used herein have the same meaning as commonly understood by one of ordinary skill in the art. Further, all terms should be interpreted as having a meaning that is consistent with their meaning in the context of the relevant art and the present disclosure and will not be interpreted in an idealized or overly formal sense unless expressly so defined herein.

The terminology used herein is for the purpose of describing particular embodiments only and is not intended to be limiting of embodiments. The singular forms “a”, “an”, and “the” are intended to include the plural forms as well, unless the context clearly indicates otherwise. The terms “comprises”, “comprising”, “includes”, “including”, “has”, “having” and any other variations thereof specify the presence of the stated features, steps, operations, elements, components and/or groups, but do not preclude the presence or addition of one or more other features, steps, operations, elements, components and/or groups thereof.

Example embodiments may be described herein with reference to the accompanying drawings. Many different forms and embodiments are possible without deviating from the teachings of the present disclosure. Accordingly, the present disclosure should not be construed as limited to the example embodiments set forth herein. As such, it will be understood by those of ordinary skill in the art that various changes in form and details may be made therein without departing from the scope of the present disclosure. The present specification, including the drawings, shall be construed to constitute a complete written description of all combinations and subcombinations of the embodiments described herein, and of the manner and process of making and using them, and shall support claims to any such combination or subcombination.

The example embodiments are mainly described in terms of particular methods and devices provided in particular implementations. However, the methods and devices may operate effectively in other implementations. Phrases such as “example embodiment”, “one embodiment”, “embodiment” and “another embodiment” may refer to the same or different embodiments as well as to multiple embodiments. The embodiments are described with respect to systems and/or devices having certain components. However, the systems and/or devices may include fewer or additional components than those shown, and variations in the arrangement and type of the components may be made without departing from the scope of the present disclosure.

The example embodiments are also described in the context of particular methods having certain steps or operations. However, the methods and devices may operate effectively for other methods having different and/or additional steps/operations and steps/operations in different orders that are not inconsistent with the example embodiments. Thus, the present disclosure is not intended to be limited to the embodiments shown, but is to be accorded the widest scope consistent with the principles and features described herein.

As will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon.

Any combination of one or more computer readable media may be utilized. The computer readable media may be a computer readable signal medium or a computer readable storage medium. A computer readable storage medium may be, for example, but is not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Aspects of the present disclosure may be described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified in the flowchart and/or block diagram block or blocks. As used herein, “a processor” may refer to one or more processors.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The flowchart and block diagrams in the figures illustrate the architecture, functionality, and operation of possible implementations of systems, methods and computer program products according to various aspects of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. Although some of the diagrams include arrows on communication paths to show a primary direction of communication, it is to be understood that communication may occur in the opposite direction to the depicted arrows. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

Computer program code for carrying out operations for aspects of the present disclosure may be written in any combination of one or more programming languages, including an object oriented programming language such as Java, JavaScript, Scala, Smalltalk, Eiffel, JADE, Emerald, C++, C#, VB.NET, JSON, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider) or in a cloud computing environment or offered as a service such as a Software as a Service (SaaS).

The above-disclosed subject matter is to be considered illustrative, and not restrictive, and the appended claims are intended to cover all such modifications, enhancements, and other embodiments, which fall within the scope of the present disclosure. Thus, to the maximum extent allowed by law, the scope is to be determined by the broadest permissible interpretation of the following claims and their equivalents, and shall not be restricted or limited by the foregoing detailed description.

1. A method for transiently enhancing transport of molecules across a cell membrane, the method comprising:
coupling one or more electrodes to target cells or tissue;
administering one or more target molecules to an environment surrounding the target cells or tissue; and
delivering an asymmetric charge-balanced electrical waveform to the target cells or tissue via the one or more electrodes, the asymmetric charge-balanced electrical waveform comprising a plurality of electrical pulses,
wherein respective ones of the electrical pulses comprise first and second phases separated by an interphase delay, the first and second phases having opposite polarities,
wherein successive ones of the electrical pulses are separated by an interpulse delay, and
wherein at least some of the first and second phases are transiently asymmetric but result in a net charge balance for the asymmetric charge-balanced electrical waveform by an end of delivery.

2. (canceled)

3. The method of claim 1, wherein the respective ones of the electrical pulses comprise bipolar square waves,
wherein a respective duration of the first phase is between 10 nanoseconds and 10 microseconds, and
wherein a respective duration of the second phase is between 10 nanoseconds and 10 microseconds.

4. The method of claim 3, wherein the respective duration of the first phase and the respective duration of the second phase are each between 0.5 and 2 microseconds, and
wherein the interphase delay is between 0.5 and 2 microseconds.

5. The method of claim 1, wherein delivery of the asymmetric charge-balanced electrical waveform induces reversible electroporation in the target cells or tissue, and
wherein at least some of the first and second phases are different in duration and/or magnitude.

6. The method of claim 1, further comprising:
placing one or more sensing probes in proximity to the one or more electrodes; and
adjusting the interpulse delay between successive ones of the electrical pulses while delivering the asymmetric charge-balanced electrical waveform to the target cells or tissue, based on data from the one or more sensing probes,
wherein the one or more sensing probes measure a temperature of the target cells or tissue, and
wherein the interpulse delay is adjusted based on the data from the one or more sensing probes to elevate the temperature of the target cells or tissue above 37° C. but below 45° C.

7. (canceled)

8. The method of claim 1, wherein the one or more target molecules comprise at least one of a recombinant nucleic acid, a nucleic acid intended for the target cells or tissue to manufacture an antigen which acts as a vaccine, a nanoparticle, a therapeutic drug substance, an immunotherapy drug substance, or a gene therapy drug substance.

9. The method of claim 1, wherein the one or more target molecules comprise a nucleic acid that results in manufacture of a therapeutic drug substance responsive to transport across a cell membrane of the target cells or tissue.

10. The method of claim 1, wherein the one or more target molecules comprise multiple different nucleic acids which result in production of molecules that cannot be created from a single nucleic acid responsive to transport across a cell membrane of the target cells or tissue.

11. The method of claim 1, wherein transport of the one or more target molecules across a cell membrane of the target cells or tissue results in a biological manufacturing of at least one of:
a virus-like particle (VLP);
a protein-based therapeutic substance comprising at least one of a protein, a peptide, an antibody, or an enzyme;
a nucleic acid-based drug substance comprising at least one of DNA, a plasmid, an oligonucleotide, an aptamer, a DNAzyme, an RNA aptamer, an RNA decoy, a microRNA fragment, or a small interfering RNA fragment; or
a gene therapy drug substance.

12. The method of claim 1, wherein a respective duration of the first phase is at least twice as long as a respective duration of the second phase, and
wherein the respective duration of the first phase is at least twice as long as a respective duration of the interphase delay.

13. The method of claim 1, wherein a respective duration of the first phase is longer than a respective duration of the second phase.

14. The method of claim 13, wherein the respective duration of the first phase is 2 microseconds or less, and
wherein the respective duration of the second phase is 1 microsecond or less.

15. The method of claim 13, wherein a respective duration of the interphase delay is 1 microsecond or less.

16. The method of claim 13, wherein a respective duration of the interphase delay is substantially equal to the respective duration of the second phase.

17. The method of claim 1, wherein the asymmetric charge-balanced electrical waveform comprises a sinusoidal waveform including sinusoidal electrical pulses, and
wherein the first and second phases of a first one of the electrical pulses are sinusoidal phases that have different durations and a same magnitude.

18. The method of claim 1, wherein the asymmetric charge-balanced electrical waveform comprises a sinusoidal waveform including sinusoidal electrical pulses,
wherein the first phase of a first one of the electrical pulses is followed by the second phase of the first one of the electrical pulses,
wherein the first phase of a second one of the electrical pulses is followed by the second phase of the second one of the electrical pulses,
wherein the second phase of the first one of the electrical pulses is followed by the first phase of the second one of the electrical pulses, without any remaining ones of the electrical pulses therebetween, and
wherein the second phase of the first one of the electrical pulses has a same magnitude as the first phase of the second one of the electrical pulses.

19. The method of claim 18, wherein the second phase of the first one of the electrical pulses has a same duration as the first phase of the second one of the electrical pulses.

20. The method of claim 18, wherein the second phase of the first one of the electrical pulses has a different duration from the first phase of the second one of the electrical pulses.

21. The method of claim 1, wherein the asymmetric charge-balanced electrical waveform comprises at least one of a ramp waveform including ramp electrical pulses, an exponential decay waveform including exponential decay electrical pulses, or a sinusoidal waveform including both a sinusoidal electrical pulse and a trapezoidal electrical pulse.

22. (canceled)

23. An apparatus configured to transiently enhance transport of molecules across a cell membrane, the apparatus comprising:
one or more electrodes configured to deliver an asymmetric charge-balanced electrical waveform to target cells or tissue, the asymmetric charge-balanced electrical waveform comprising a plurality of electrical pulses;
a power supply configured to output the electrical pulses to the one or more electrodes; and
a controller communicatively coupled with the power supply and configured to control the power supply to output the electrical pulses,
wherein respective ones of the electrical pulses comprise first and second phases separated by an interphase delay, the first and second phases having opposite polarities,
wherein successive ones of the electrical pulses are separated by an interpulse delay, and
wherein at least some of the first and second phases are transiently asymmetric but result in a net charge balance for the asymmetric charge-balanced electrical waveform by an end of delivery.

24-25. (canceled)
